(19) United States
(12) Patent Application Publication
(10) Pub. No.: US 2025/0177352 A1
(43) Pub. Date: Jun. 5, 2025

(54) COMBINATION TREATMENT FOR CANCER

(71) Applicant: LAEKNA LIMITED, Hong Kong (CN)

(72) Inventors: Chris LU, Lexington, MA (US); Yong YUE, Warren, NJ (US); Wenyue MA, Beijing (CN); Juan LIU, Shanghai (CN); Yan CHEN, Shanghai (CN)

(21) Appl. No.: 18/845,159

(22) PCT Filed: Mar. 13, 2023
(86) PCT No.: PCT/CN2023/081086

§ 371 Date: Sep. 9, 2024

(30) Foreign Application Priority Data
Mar. 14, 2022 (WO) PCT/CN2022/080565

(51) Int. Cl. A61K 31/4155; A61K 39/00; A61K 39/395; A61K 45/06; A61P 35/00; C07K 16/28

(52) U.S. Cl. CPC A61K 31/4155; A61K 39/39558; A61K 45/06; A61P 35/00; C07K 16/2827; A61K 2039/505

(57) ABSTRACT

This disclosure features methods of treating cancer, such as breast cancer, in a subject, comprising administering to the subject a combination of afuresertib, an anti-PD-L1 antibody, and optionally, a chemotherapeutic agent. Dosage regimens for the combination therapy are also disclosed.

### CROSS REFERENCE TO RELATED APPLICATIONS

This application claims priority to PCT/CN2022/080565 filed Mar. 14, 2022, entitled COMBINATION TREATMENT FOR CANCER, the contents of which is incorporated by reference herein.

### SEQUENCE LISTING

The instant application contains a Sequence Listing which has been submitted electronically in ASCII format and is hereby incorporated by reference in its entirety. Said ASCII copy, created on Mar. 10, 2022, is named 45607-0013W00_SL.txt and is 36,109 bytes in size.

### FIELD OF INVENTION

The present disclosure relates to a method of treating cancer and to combinations and dosage regimens useful in such treatment.

### BACKGROUND

Effective treatment of hyperproliferative disorders including cancer is a continuing goal in the oncology field. Generally, cancer results from the deregulation of the normal processes that control cell division, differentiation and apoptotic cell death and is characterized by the proliferation of malignant cells, which have the potential for unlimited growth, local expansion and systemic metastasis. Deregulation of normal processes includes abnormalities in signal transduction pathways, and/or abnormalities in the regulation of gene transcription, and/or responses to factors (e.g., growth factors) which differ from those found in normal cells.

Solid cancers such as breast cancer (BC) is the most common malignant neoplasm in women worldwide. 268,600 new cases and 69,500 deaths occurred in China in 2015 (Chen W, et al, 2015. CA Cancer J Clin. 2016; 66(2):115-132). In the US, BC is the most common cancer in women with similar number of new cases to China and the fourth leading cause of cancer-related deaths (regardless of gender) behind lung cancer, colorectal cancer, and pancreatic cancer (Hyuna Sung et al., Ca Cancer J. Clin. 2021; 71:209-249). Triple-negative breast cancer (TNBC) accounted for 12% of breast cancers diagnosed in the United States from 2012 to 2016, with a 5-year survival 8% to 16% lower than hormone receptor-positive disease. Approximately 22.9% of BCs in China (Shan Zheng, et al., Appl Immunohistochem Mol Morphol 2014; 22(5):383-389) are TNBC and constitute an aggressive histologic subtype (Mihriban K, et al., Nature Communications volume 9, Article number: 3588 (2018); Claire H. Li, et al., Breast Cancer Res. 2019; 21(143). TNBC does not express estrogen and progesterone receptors and lack amplification/overexpression of the human epidermal growth factor 2 receptor (HER2). Because the cancer cells lack these proteins, hormone therapy and drugs that target HER2 are not helpful and chemotherapy is the main systemic treatment option. Even though TNBC tends to respond well to chemotherapy initially, it recurs more frequently than other breast cancers. Thus, the overall prognosis for patients with TNBC is worse than for other BC subtypes, and more effective and safe therapeutic options are unmet medical needs.

### SUMMARY

The present application provides a novel combination for the treatment of a cancer, e.g., a solid cancer such as triple negative breast cancer. The disclosure provides, inter alia, a method of treating cancer, (e.g., a triple negative breast cancer) in a subject, comprising administering to the subject: (i) afuresertib, or a pharmaceutically acceptable salt thereof; (ii) an isolated antibody molecule capable of binding to human Programmed Death-Ligand 1 (PD-L1); and (iii) optionally a chemotherapeutic agent such as paclitaxel or nab-paclitaxel.

The details of one or more embodiments of the invention are set forth in the description below. Other features, objects, and advantages of the invention will be apparent from the description and from the claims.

In some aspects, the disclosure features a method of treating a cancer in a subject, comprising administering to the subject patient in need thereof an effective amount of: (i)N-{(1S)-2-amino-1-[(3-fluorophenyl)methyl]ethyl}-5-chloro-4-(4-chloro-1methyl-1H-pyrazol-5-yl)-2-thiophenecarboxamide (afuresertib), or a pharmaceutically acceptable salt thereof; and (ii) and an isolated antibody molecule capable of binding to human Programmed Death-Ligand 1 (PD-L1).

In some aspects, the disclosure features the use of an effective amount of: afuresertib, or a pharmaceutically acceptable salt thereof; and (ii) and an isolated antibody molecule capable of binding to human Programmed Death-Ligand 1 (PD-L1) in any of the methods described herein. In some embodiments, the disclosure features the use of an effective amount of: afuresertib, or a pharmaceutically acceptable salt thereof; and (ii) and an isolated antibody molecule capable of binding to human Programmed Death-Ligand 1 (PD-L1) for treating cancer in a subject (e.g., a human patient).

In some aspects, the disclosure features an effective amount of: afuresertib, or a pharmaceutically acceptable salt thereof; and (ii) and an isolated antibody molecule capable of binding to human Programmed Death-Ligand 1 (PD-L1) for use in any of the methods of treatment described herein. In some aspects, the disclosure features an effective amount of: afuresertib, or a pharmaceutically acceptable salt thereof; and (ii) and an isolated antibody molecule capable of binding to human Programmed Death-Ligand 1 (PD-L1) for use in treating cancer in a subject (e.g., a human patient).

In some embodiments of any of the above aspects, the disclosure features the use of a chemotherapeutic agent in addition to the anti-PD-L1 antibody and the afuresertib in any of the methods of treatment described herein. In some embodiments of any of the above aspects, the disclosure features a chemotherapeutic agent for use in any of the methods of treatment described herein, in addition to the anti-PD-L1 antibody and the afuresertib. In some embodiments, the chemotherapeutic agent is paclitaxel or nab-paclitaxel.

In some aspects, the disclosure features a kit for the treatment of cancer, the kit comprising, in separate containers, (a) a first pharmaceutical composition comprising afuresertib, or a pharmaceutically acceptable salt thereof; and (b) a second pharmaceutical composition comprising an anti-PD-L1 antibody. In some embodiments, the kit further comprises a third composition comprising paclitaxel or nab-paclitaxel.

In some embodiments of any of the above aspects, the cancer is a breast cancer. In some embodiments, the cancer is a metastatic triple negative breast cancer.

### BRIEF DESCRIPTION OF DRAWINGS

FIG. 1 is a graph showing the cell viability of MBT-2 cells in a proliferation assay in the presence of varying concentrations of various AKT inhibitors (Afuresertib, Ipatasertib, AZD5363, MK2066) and a solvent control dimethylsulfoxide (DMSO) as indicated. The data points represent the mean luminescence, and error bars represent standard deviations (SDs).

FIG. 2A is a graph showing the cell-surface binding of PD-L1 for the indicated cell lines (Renca, Pan02 or MBT-2) in the presence of varying concentrations of anti-PD-L1 antibody as indicated on the x-axis.

FIG. 2B is an inset graph of FIG. 2A.

FIG. 3 is a graph showing changes in body weight of MBT-2 tumor-bearing mice after treatment with each compound. The data points represent the mean weight of each group, and error bars represent SEMs. Afure: Afuresertib; PO: oral administration; QD: once a day administration, BIW: twice a week administration; IP: intraperitoneal.

FIG. 4 is a graph showing relative body weight changes calculated based on animal weight at the start of administration. The data points represent percent changes in mean weight of each group, and error bars represent SEMs. Afure: Afuresertib; PO: oral administration; QD: once a day administration, BIW: twice a week administration; IP: intraperitoneal.

FIG. 5 is a graph showing tumor growth curves for MBT-2 tumor-bearing mice after treatment with each compound. The data points represent mean tumor volumes in various groups, and error bars represent SEMs. Afure: Afuresertib

FIG. 6 is a graph showing survival curves of MBT-2 tumor-bearing mice after treatment. Afure: Afuresertib; PO: oral administration; QD: once a day administration, BIW: twice a week administration; IP: intraperitoneal.

FIG. 7 is a scheme showing the Phase 1 dose escalation. QD: once a day administration, Q3W: once in three weeks administration; DLT: Dose limiting toxicity.

FIG. 8 is a scheme showing the Phase I and Phase II study diagram. IV: intravenous; QD: once a day administration, Q3W: once in three weeks administration; DLT: Dose limiting toxicity. BOIN Design: Bayesian Optimal Interval Design.

FIG. 9 is a flowchart showing imaging and treatment for clinically stable participants treated with anti-PD-L1 Ab after first radiologic evidence of progressive disease (PD) assessed by the investigator. iRECIST: immune Response Evaluation Criteria in Solid Tumours.

### DETAILED DESCRIPTION

The present disclosure relates to methods for treating cancer, e.g., triple negative breast cancer (TNBC) using a combination of (i) afuresertib, or a pharmaceutically acceptable salt thereof; (ii) an isolated antibody molecule capable of binding to human Programmed Death-Ligand 1 (PD-L1); and (iii) optionally a chemotherapeutic agent such as paclitaxel or nab-paclitaxel.

### Afuresertib

Afuresertib is an orally bioavailable inhibitor of the serine/threonine protein kinase Akt (protein kinase B) with potential antineoplastic activity. It is known to be a reversible, ATP-competitive, oral, low-nanomolar, pan-AKT kinase inhibitor. Afuresertib binds to and inhibits the activity of Akt, which may result in inhibition of the PI3K/Akt signaling pathway and tumor cell proliferation and the induction of tumor cell apoptosis. Afuresertib has the following chemical structure:

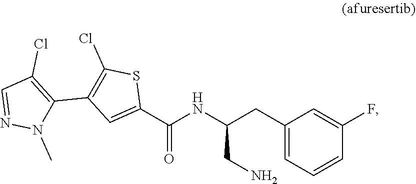 [SMILES: Cn1ncc(Cl)c1-c1cc(C(=O)N[C@H](CN)Cc2cccc(F)c2)sc1Cl]

and chemical name: N-{(1S)-2-amino-1-[(3-fluorophenyl)methyl]ethyl}-5-chloro-4-(4-chloro-1-methyl-1H-pyrazol-5-yl)-2-thiophenecarboxamide.

Afuresertib and the methods of making afuresertib have been described in detail in International Patent Publication Nos. WO2008098104 WO201008833, WO2015049649, and WO2021026454, the disclosures of each of which is incorporated herein by reference in its entirety.

In some embodiments, afuresertib is in the form of a hydrochloride salt. In some embodiments, afuresertib is in the form of a hydrochloride salt having e.g., a 1:1 stoichiometric ratio of N-{(1S)-2-amino-1-[(3-fluorophenyl)methyl]ethyl}-5-chloro-4-(4-chloro-1-methyl-1H-pyrazol-5-yl)-2-thiophenecarboxamide to hydrochloric acid. In some embodiments, afuresertib is in the form of crystalline N-{(1S)-2-amino-1-[(3-fluorophenyl)methyl]ethyl}-5-chloro-4-(4-chloro-1-methyl-1H-pyrazol-5-yl)-2-thiophenecarboxamide hydrochloride. In some embodiments, the crystalline hydrochloride salt has one or more, two or more, three or more, four or more, five or more, or six or more characteristic diffraction peaks in terms of 2-theta (±0.3°) selected from 7.2°, 14.4°, 17.9°, 18.5°, 20.8°, 21.5°, 22.4°, 22.9°, 23.7°, 24.5°, 24.7°, 25.1°, 25.7°, 27.3°, 28.2°, 28.8°, 30.4°, 32.4°, 32.7°, 35.2°, 36.1°, 40.0°, 41.3°, and 41.7°, as measured in an Powder X-Ray Diffractogram using Cu Kα radiation. In some embodiments, the crystalline hydrochloride salt has a DSC thermogram having an endothermic peak at about 220° C. Methods of making crystalline afuresertib and salts thereof are described in U.S. Pat. No. 8,609,711, the content of which is incorporated herein in its entirety.

In some embodiments, the afuresertib, or a pharmaceutically acceptable salt thereof, is administered to the patient once daily (QD). In some embodiments, afuresertib, or a pharmaceutically acceptable salt thereof, is administered to the patient in a dosage of from about 75 mg to about 150 mg, on a free base basis, once per day. In some embodiments, afuresertib, or a pharmaceutically acceptable salt thereof, is administered to the patient in a dosage of about 10 mg, about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, or about 150 mg, on a free base basis, once per day. In some embodiments, afuresertib, or a pharmaceutically acceptable salt thereof, is administered to the patient in a dosage of about 75 mg, about 100 mg, about 125 mg or about 150 mg, on a free base basis, once per day.

In some embodiments, afuresertib, or a pharmaceutically acceptable salt thereof, is administered to the patient in a dosage of about 10 mg, on a free base basis, once per day. In some embodiments, afuresertib, or a pharmaceutically acceptable salt thereof, is administered to the patient in a dosage of about 20 mg, on a free base basis, once per day. In some embodiments, afuresertib, or a pharmaceutically acceptable salt thereof, is administered to the patient in a dosage of about 25 mg, on a free base basis, once per day. In some embodiments, afuresertib, or a pharmaceutically acceptable salt thereof, is administered to the patient in a dosage of about 30 mg, on a free base basis, once per day. In some embodiments, afuresertib, or a pharmaceutically acceptable salt thereof, is administered to the patient in a dosage of about 40 mg, on a free base basis, once per day. In some embodiments, afuresertib, or a pharmaceutically acceptable salt thereof, is administered to the patient in a dosage of about 50 mg, on a free base basis, once per day. In some embodiments, afuresertib, or a pharmaceutically acceptable salt thereof, is administered to the patient in a dosage of about 60 mg, on a free base basis, once per day. In some embodiments, afuresertib, or a pharmaceutically acceptable salt thereof, is administered to the patient in a dosage of about 70 mg, on a free base basis, once per day. In some embodiments, afuresertib, or a pharmaceutically acceptable salt thereof, is administered to the patient in a dosage of about 75 mg, on a free base basis, once per day. In some embodiments, afuresertib, or a pharmaceutically acceptable salt thereof, is administered to the patient in a dosage of about 80 mg, on a free base basis, once per day. In some embodiments, afuresertib, or a pharmaceutically acceptable salt thereof, is administered to the patient in a dosage of about 90 mg, on a free base basis, once per day. In some embodiments, afuresertib, or a pharmaceutically acceptable salt thereof, is administered to the patient in a dosage of about 100 mg, on a free base basis, once per day. In some embodiments, afuresertib, or a pharmaceutically acceptable salt thereof, is administered to the patient in a dosage of about 110 mg, on a free base basis, once per day. In some embodiments, afuresertib, or a pharmaceutically acceptable salt thereof, is administered to the patient in a dosage of about 120 mg, on a free base basis, once per day. In some embodiments, afuresertib, or a pharmaceutically acceptable salt thereof, is administered to the patient in a dosage of about 125 mg, on a free base basis, once per day. In some embodiments, afuresertib, or a pharmaceutically acceptable salt thereof, is administered to the patient in a dosage of about 130 mg, on a free base basis, once per day. In some embodiments, afuresertib, or a pharmaceutically acceptable salt thereof, is administered to the patient in a dosage of about 140 mg, on a free base basis, once per day. In some embodiments, afuresertib, or a pharmaceutically acceptable salt thereof, is administered to the patient in a dosage of about 150 mg, on a free base basis, once per day.

In some embodiments, the afuresertib inhibits AKT (protein kinase B) activity and/or tumor cell proliferation as measured in in vitro assays known in the art.

### Anti-PD-L1 Antibody

PD-L1 (also known as cluster of differentiation 274 (CD274) or B7 homolog 1 (B7-H1)) is a 40 kDa type 1 transmembrane protein. PD-L1 binds to its receptor, PD-1, found on activated T cells, B cells, and myeloid cells, to modulate activation or inhibition. Both PD-L1 and PD-L2 are B7 homologs that bind to PD-1, but do not bind to CD28 or CTLA-4 (Blank et al. (2005) Cancer Immunol Immunother. 54:307-14). Binding of PD-L1 with its receptor PD-1 on T cells delivers a signal that inhibits TCR-mediated activation of IL-2 production and T cell proliferation. The mechanism involves inhibition of ZAP70 phosphorylation and its association with CD3 ζ (Sheppard et al. (2004) FEBS Lett. 574:37-41). PD-1 signaling attenuates PKC-θ activation loop phosphorylation resulting from TCR signaling, necessary for the activation of transcription factors NF-κB and AP-1, and for production of IL-2. PD-L1 also binds to the costimulatory molecule CD80 (B7-1), but not CD86 (B7-2) (Butte et al. (2008) Mol Immunol. 45:3567-72).

Expression of PD-L1 on the cell surface has been shown to be upregulated through IFN-γ stimulation. PD-L1 expression has been found in many cancers, including human lung, ovarian and colon carcinoma and various myelomas, and is often associated with poor prognosis (Iwai et al. (2002) PNAS 99:12293-7; Ohigashi et al. (2005) Clin Cancer Res 11:2947-53; Okazaki et al. (2007) Intern. Immun. 19:813-24; Thompson et al. (2006) Cancer Res. 66:3381-5). PD-L1 has been suggested to play a role in tumor immunity by increasing apoptosis of antigen-specific T-cell clones (Dong et al. (2002) Nat Med 8:793-800). It has also been suggested that PD-L1 might be involved in intestinal mucosal inflammation and inhibition of PD-L1 suppresses wasting disease associated with colitis (Kanai et al. (2003) J Immunol 171:4156-63).

In some embodiments, the anti-PD-L1 antibody used in the methods herein has demonstrated a preliminary anti-tumor effects and a good safety profile in patients with advanced solid tumors from the phase I first-in-human clinical study (FAZ052(LAE005) Investigator's Brochure, Edition 5. Novartis Institutes for BioMedical Research; Filip Janku, et al. Presented at the Society for Immunotherapy of Cancer (SITC) 33rd Annual Meeting; Nov. 7-11, 2018; Washington, D.C.; Interim analysis of Novartis phase I study of LAE005 in patients with solid tumor. Novartis Institutes for BioMedical Research). In an ongoing phase I study with the anti-PD-L1 antibody monotherapy in 93 subjects with advanced solid tumors including mTNBC, the ORR of 6.0% treated with the anti-PD-L1 antibody 1200 mg IV Q3W is considered comparable to the ORRs of other approved anti-PD-L1 antibodies from Phase I studies in solid tumors, such as Avelumab (ORR 9%) (Hassan R, et al. JAMA Oncol. 2019, 1; 5(3):351-357), Pembrolizumab (ORR 5.3%) (Adams S, et al., Ann Oncol. 2019 1; 30(3):397-404) and Atezolizumab (ORR 10%) (Emens L A, Cruz C, Eder J P, et al. et al JAMA Oncol. 2019; 5:74).

In some embodiments, the anti-PD-L1 antibody of the disclosure is any anti-PD-L1 antibody described in International Patent Application No. WO2016061142 and WO2019200229, each of which is incorporated herein in its entirety.

In some embodiments, the anti-PD-L1 antibody of the disclosure has the following six complementarity determining regions (CDR) sequences (SEQ ID NOs.: 1-6), as shown below in Table 1, which correspond to SEQ ID NOs: 195, 2, 3, and 9-11 in WO2016061142. In some embodiments, the CDRs are according to the Kabat definition. In some embodiments, the CDRs are according to the Chothia definition. In some embodiments, the CDRs are according to the combined CDR definitions of both Kabat and Chothia. In one embodiment, the combination of Kabat and Chothia CDR of the variable heavy (VH) chain CDR1 comprises the amino acid sequence GYTFTSYWMY (SEQ ID NO: 1).

TABLE 1
anti-PD-L1 antibody CDR sequences

| SEQ ID NO: | Type of sequence | Sequence |
|---|---|---|
| 1 | Heavy chain CDR1 | GYTFTSYWMY |
| 2 | Heavy chain CDR2 | RIDPNSGSTKYNEKFKN |
| 3 | Heavy chain CDR3 | DYRKGLYAMDY |
| 4 | Light chain CDR1 | KASQDVGTAVA |
| 5 | Light chain CDR2 | WASTRHT |
| 6 | Light chain CDR3 | QQYNSYPLT |

In some embodiments, the antibody has any one of the following heavy CDR1 sequences as shown in Table 2 below (SEQ ID NOs. 1, 7, or 9), along with the indicated HCDR2 (SEQ ID NO: 2 or 8), HCDR3 (SEQ ID NO: 3), and LCDR1-3 sequences (SEQ ID NOs.: 10-12).

TABLE 2
anti-PD-L1 antibody CDR sequences

| SEQ ID NO: | Type of sequence | Sequence |
|---|---|---|
| 7 | Heavy chain CDR1 | SYWMY |
| 2 | Heavy chain CDR2 | RIDPNSGSTKYNEKFKN |
| 8 | Heavy chain CDR2 | DPNSGS |
| 3 | Heavy chain CDR3 | DYRKGLYAMDY |
| 9 | Heavy chain CDR1 | GYTFTSY |
| 1 | Heavy chain CDR1 | GYTFTSYWMY |
| 10 | Light chain CDR1 | SQDVGTA |
| 11 | Light chain CDR2 | WAS |
| 12 | Light chain CDR3 | YNSYPL |

In some embodiments, the anti-PD-L1 antibody of the disclosure has the following VH, VL, Heavy and Light chain sequences as shown in Table 3 corresponding to SEQ ID NOs: 18, 20, 22, 24, 30, 32, 34, 36, 38, 42, 44, 78, 80, 82, 84, and 91 in WO2016061142.

TABLE 3
anti-PD-L1 antibody VH, VL, HC, LC sequences

| | Type of | |
|---|---|---|
| SEQ ID NO: | sequence | Sequence |
| 13 | Variable | EVQLVQSGAEVKKPGATVKISCKVSGYTFTSYWMYWVRQAR |
| | Heavy (VH) | GQRLEWIGRIDPNSGSTKYNEKFKNRFTISRDNSKNTLYLQMN |
| | | SLRAEDTAVYYCARDYRKGLYAMDYWGQGTTVTVSS |
| 14 | Variable | QVQLVQSGAEVKKPGASVKVSCKASGYTFTSYWMYWVRQAT |
| | Heavy (VH) | GQGLEWMGRIDPNSGSTKYNEKFKNRFTISRDDSKNTAYLQM |
| | | NSLKTEDTAVYYCARDYRKGLYAMDYWGQGTTVTVSS |
| 15 | Variable | EVQLVQSGAEVKKPGATVKISCKVSGYTFTSYWMYWVRQAT |
| | Heavy (VH) | GQGLEWMGRIDPNSGSTKYNEKFKNRVTITADKSTSTAYMELS |
| | | SLRSEDTAVYYCARDYRKGLYAMDYWGQGTTVTVSS |
| 16 | Variable | EVQLVQSGAEVKKPGESLRISCKGSGYTFTSYWMYWVRQAPG |
| | Heavy (VH) | QGLEWMGRIDPNSGSTKYNEKFKNRVTISVDTSKNQFSLKLSS |
| | | VTAADTAVYYCARDYRKGLYAMDYWGQGTTVTVSS |
| 17 | Variable | AIQLTQSPSSLSASVGDRVTITCKASQDVGTAVAWYLQKPGQS |
| | Light (VL) | PQLLIYWASTRHTGVPSRFSGSGSGTDFTFTISSLEAEDAATYY |
| | | CQQYNSYPLTFGQGTKVEIK |
| 18 | Variable | DIVMTQTPLSLPVTPGEPASISCKASQDVGTAVAWYLQKPGQS |
| | Light (VL) | PQLLIYWASTRHTGIPARFSGSGSGTEFTLTISSLQSEDFAVYYC |
| | | QQYNSYPLTFGQGTKVEIK |
| 19 | Variable | EIVLTQSPDFQSVTPKEKVTITCKASQDVGTAVAWYLQKPGQS |
| | Light (VL) | PQLLIYWASTRHTGVPDRFSGSGSGTDFTLKISRVEAEDVGVY |
| | | YCQQYNSYPLTFGQGTKVEIK |
| 20 | Variable | EIVLTQSPDFQSVTPKEKVTITCKASQDVGTAVAWYLQKPGQS |
| | Light (VL) | PQLLIYWASTRHTGVPSRFSGSGSGTDFTFTISSLQPEDIATYYC |
| | | QQYNSYPLTFGQGTKVEIK |
| 21 | Heavy Chain | EVQLVQSGAEVKKPGATVKISCKVSGYTFTSYWMYWVRQAR |
| | | GQRLEWIGRIDPNSGSTKYNEKFKNRFTISRDNSKNTLYLQMN |
| | | SLRAEDTAVYYCARDYRKGLYAMDYWGQGTTVTVSSASTKG |
| | | PSVFPLAPCSRSTSESTAALGCLVKDYFPEPVTVSWNSGALTSG |
| | | VHTFPAVLQSSGLYSLSSVVTVPSSSLGTKTYTCNVDHKPSNTK |
| | | VDKRVESKYGPPCPPCPAPEFLGGPSVFLFPPKPKDTLMISRTPE |
| | | VTCVVVDVSQEDPEVQFNWYVDGVEVHNAKTKPREEQFNST |
| | | YRVVSVLTVLHQDWLNGKEYKCKVSNKGLPSSIEKTISKAKG |
| | | QPREPQVYTLPPSQEEMTKNQVSLTCLVKGFYPSDIAVEWESN |
| | | GQPENNYKTTPPVLDSDGSFFLYSRLTVDKSRWQEGNVFSCSV |
| | | MHEALHNHYTQKSLSLSLG |
| 22 | Heavy Chain | QVQLVQSGAEVKKPGASVKVSCKASGYTFTSYWMYWVRQAT |
| | | GQGLEWMGRIDPNSGSTKYNEKFKNRFTISRDDSKNTAYLQM |
| | | NSLKTEDTAVYYCARDYRKGLYAMDYWGQGTTVTVSSASTK |
| | | GPSVFPLAPCSRSTSESTAALGCLVKDYFPEPVTVSWNSGALTS |
| | | GVHTFPAVLQSSGLYSLSSVVTVPSSSLGTKTYTCNVDHKPSNT |
| | | KVDKRVESKYGPPCPPCPAPEFLGGPSVFLFPPKPKDTLMISRT |
| | | PEVTCVVVDVSQEDPEVQFNWYVDGVEVHNAKTKPREEQFNS |
| | | TYRVVSVLTVLHQDWLNGKEYKCKVSNKGLPSSIEKTISKAKG |
| | | QPREPQVYTLPPSQEEMTKNQVSLTCLVKGFYPSDIAVEWESN |
| | | GQPENNYKTTPPVLDSDGSFFLYSRLTVDKSRWQEGNVFSCSV |
| | | MHEALHNHYTQKSLSLSLGK |
| 23 | Heavy Chain | EVQLVQSGAEVKKPGATVKISCKVSGYTFTSYWMYWVRQAT |
| | | GQGLEWMGRIDPNSGSTKYNEKFKNRVTITADKSTSTAYMELS |
| | | SLRSEDTAVYYCARDYRKGLYAMDYWGQGTTVTVSSASTKG |
| | | PSVFPLAPCSRSTSESTAALGCLVKDYFPEPVTVSWNSGALTSG |
| | | VHTFPAVLQSSGLYSLSSVVTVPSSSLGTKTYTCNVDHKPSNTK |
| | | VDKRVESKYGPPCPPCPAPEFLGGPSVFLFPPKPKDTLMISRTPE |
| | | VTCVVVDVSQEDPEVQFNWYVDGVEVHNAKTKPREEQFNST |
| | | YRVVSVLTVLHQDWLNGKEYKCKVSNKGLPSSIEKTISKAKG |
| | | QPREPQVYTLPPSQEEMTKNQVSLTCLVKGFYPSDIAVEWESN |
| | | GQPENNYKTTPPVLDSDGSFFLYSRLTVDKSRWQEGNVFSCSV |
| | | MHEALHNHYTQKSLSLSLGK |
| 24 | Heavy Chain | EVQLVQSGAEVKKPGESLRISCKGSGYTFTSYWMYWVRQAPG |
| | | QGLEWMGRIDPNSGSTKYNEKFKNRVTISVDTSKNQFSLKLSS |
| | | VTAADTAVYYCARDYRKGLYAMDYWGQGTTVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEV |
| | | TCVVVDVSQEDPEVQFNWYVDGVEVHNAKTKPREEQFNSTY |
| | | RVVSVLTVLHQDWLNGKEYKCKVSNKGLPSSIEKTISKAKGQP |
| | | REPQVYTLPPSQEEMTKNQVSLTCLVKGFYPSDIAVEWESNGQ |
| | | PENNYKTTPPVLDSDGSFFLYSRLTVDKSRWQEGNVFSCSVMH |
| | | EALHNHYTQKSLSLSLG |
| 25 | Light chain | AIQLTQSPSSLSASVGDRVTITCKASQDVGTAVAWYLQKPGQS |
| | | PQLLIYWASTRHTGVPSRFSGSGSGTDFTFTISSLEAEDAATYY |
| | | CQQYNSYPLTFGQGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 26 | Light chain | DIVMTQTPLSLPVTPGEPASISCKASQDVGTAVAWYLQKPGQS |
| | | PQLLIYWASTRHTGIPARFSGSGSGTEFTLTISSLQSEDFAVYYC |
| | | QQYNSYPLTFGQGTKVEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| 27 | Light chain | EIVLTQSPDFQSVTPKEKVTITCKASQDVGTAVAWYLQKPGQS |
| | | PQLLIYWASTRHTGVPDRFSGSGSGTDFTLKISRVEAEDVGVY |
| | | YCQQYNSYPLTFGQGTKVEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGEC |
| 28 | Light chain | EIVLTQSPDFQSVTPKEKVTITCKASQDVGTAVAWYLQKPGQS |
| | | PQLLIYWASTRHTGVPSRFSGSGSGTDFTFTISSLQPEDIATYYC |
| | | QQYNSYPLTFGQGTKVEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |

In some embodiments, the anti-PD-L1 antibody molecule comprises a VH comprising the amino acid sequence of any one of SEQ ID NOs: 13-16, or an amino acid sequence at least 85%, 90%, 95%, or 99% identical to any one of SEQ ID NOs: 13-16. In some embodiments, the anti-PD-L1 antibody molecule comprises a VL comprising the amino acid sequence of any one of SEQ ID NOs: 17-20, or an amino acid sequence at least 85%, 90%, 95%, or 99% identical to any one of SEQ ID NOs: 17-20. In some embodiments, the anti-PD-L1 antibody molecule comprises a heavy chain comprising the amino acid sequence of any one of SEQ ID NOs: 21-24, or an amino acid sequence at least 85%, 90%, 95%, or 99% identical to any one of SEQ ID NOs: 21-24. In some embodiments, the anti-PD-L1 antibody molecule comprises a light chain comprising the amino acid sequence of any one of SEQ ID NOs: 25-28, or an amino acid sequence at least 85%, 90%, 95%, or 99% identical to any one of SEQ ID NOs: 25-28.

In some embodiments the anti-PD-L1 antibody has a heavy chain variable domain comprising the amino acid sequence of SEQ ID NO: 13 and a light chain variable domain comprising the amino acid sequence of SEQ ID NO: 17. In some embodiments the anti-PD-L1 antibody has a heavy chain variable domain comprising the amino acid sequence of SEQ ID NO: 14 and a light chain variable domain comprising the amino acid sequence of SEQ ID NO: 18. In some embodiments the anti-PD-L1 antibody has a heavy chain variable domain comprising the amino acid sequence of SEQ ID NO: 15 and a light chain variable domain comprising the amino acid sequence of SEQ ID NO: 19. In some embodiments the anti-PD-L1 antibody has a heavy chain variable domain comprising the amino acid sequence of SEQ ID NO: 16 and a light chain variable domain comprising the amino acid sequence of SEQ ID NO: 20.

In some embodiments the anti-PD-L1 antibody has a heavy chain comprising the amino acid sequence of SEQ ID NO: 21 and a light chain comprising the amino acid sequence of SEQ ID NO: 25. In some embodiments the anti-PD-L1 antibody has a heavy chain comprising the amino acid sequence of SEQ ID NO: 22 and a light chain comprising the amino acid sequence of SEQ ID NO: 26. In some embodiments the anti-PD-L1 antibody has a heavy chain comprising the amino acid sequence of SEQ ID NO: 23 and a light chain comprising the amino acid sequence of SEQ ID NO: 27. In some embodiments the anti-PD-L1 antibody has a heavy chain comprising the amino acid sequence of SEQ ID NO: 24 and a light chain comprising the amino acid sequence of SEQ ID NO: 28.

In some embodiments, the anti-PD-L1 antibody is administered to the subject once every three weeks at a dosage of about 500 mg to about 2000 mg; about 500 mg to about 1500 mg; about 800 mg to about 1200 mg; about 800 mg; about 900 mg; about 1000 mg; about 1100 mg; or about 1200 mg. In some embodiments, the anti-PD-L1 antibody is administered to the patient in a dosage of about 800 mg once every 3 weeks. In some embodiments, the anti-PD-L1 antibody is administered to the patient in a dosage of about 900 mg once every 3 weeks. In some embodiments, the anti-PD-L1 antibody is administered to the patient in a dosage of about 1000 mg once every 3 weeks. In some embodiments, the anti-PD-L1 antibody is administered to the patient in a dosage of about 1100 mg once every 3 weeks. In some embodiments, the anti-PD-L1 antibody is administered to the patient in a dosage of about 1200 mg once every 3 weeks.

In some embodiments, the anti-PD-L1 antibody is any anti-PD-L1 antibody known in the art, including, but not limited to Atezolizumab, Avelumab, and Durvalumab. In one embodiment, the anti-PD-L1 antibody molecule is Atezolizumab (Genentech/Roche), also known as MPDL3280A, RG7446, R05541267, YW243.55.S70, or TECENTRIQ™ Atezolizumab and other anti-PD-L1 antibodies are disclosed in U.S. Pat. No. 8,217,149, incorporated by reference in its entirety. In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In some embodiments, the anti-PD-L1 antibody is LAE005(FAZ052).

Further anti-PD-L1 antibodies include those described, e.g., in WO 2015/181342, WO2014/100079, WO 2016/000619, WO 2014/022758, WO 2014/055897, WO 2015/061668, WO2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In some embodiments, the anti-PD-L1 antibody is an antibody that competes for binding with, and/or binds to the same epitope on PD-L1 as, one of the anti-PD-L1 antibodies described herein.

In some embodiments, the anti-PD-L1 antibody has one or more of the following functions as demonstrated in in vitro assays known in the art: (a) inhibits binding of PD-1 to PD-L1 in cells that express PD-L1; (b) inhibits PD-L1-associated intracellular signaling; (c) inhibits tumor cell proliferation; (d) enhances anti-tumor cytokine secretion; and (e) enhances T-cell proliferation.

### Nab-Paclitaxel

In some embodiments of the disclosure, the treatment further comprises administering a chemotherapeutic agent in combination with the afuresertib and the anti-PD-L1 antibody. The chemotherapeutic agent can be a platinum-based agent, a taxane, an epothilone, an anti-microtubule agent, an immunomodulatory agent, and a proteasome inhibitor. In some embodiments, the chemotherapeutic agent is paclitaxel or nab-paclitaxel.

Without being bound any particular theory, it is believed that paclitaxel is an anti-microtubule agent that promotes the assembly of microtubules from tubulin dimers and stabilizes microtubules by preventing depolymerization. This stability can result in the inhibition of the normal dynamic reorganization of the microtubule network that is essential for vital interphase and mitotic cellular functions. In addition, paclitaxel can induce abnormal arrays or “bundles” of microtubules throughout the cell cycle and multiple asters of microtubules during mitosis. Paclitaxel can be obtained via a semi-synthetic process from Taxus baccata. Nab-paclitaxel is a nanoparticle albumin-bound paclitaxel, which is superior to paclitaxel in PK and pharmacodynamics and thus has better efficacy and safety. Nab-paclitaxel has been approved for the treatment of multiple solid tumors including mTNBC. Paclitaxel has the chemical formula C₄₇H₅₁NO₁₄, the TUPAC name [(1S,2S,3R,4S,7R,9S,10S,12R,15S)-4,12-diacetyloxy-15-[(2R,3S)-3-benzamido-2-hydroxy-3-phenylpropanoyl]oxy-1,9-dihydroxy-10,14,17,17-tetramethyl-11-oxo-6-oxatetracyclo[11.3.1.0^{3,10}.0⁴⁻⁷]heptadec-13-en-2-yl]benzoate, and the following structure:

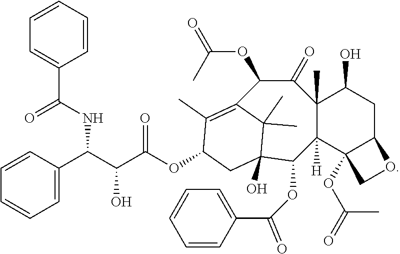 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Nab-paclitaxel and paclitaxel are described in U.S. Pat. Nos. 6,753,006, 7,758,891, 8,314,156, 8,268,348, 8,138,229, 8,034,375, 7,923,536, and 7,820,788, all of which are incorporated herein by reference.

### Cancer

The term “cancer” refers to the physiological condition in mammals that is typically characterized by unregulated cell growth/proliferation, and includes all types of cancer, neoplasm or malignant tumors found in mammals (e.g., humans), including leukemia (such as acute or chronic leukemia), carcinomas and sarcomas. In some embodiments, cancers that are treatable in the methods described herein include, for example, any solid tumors, e.g., breast cancer, prostate cancer, liver cancer, lung cancer, pancreatic cancer, renal cell carcinoma, bladder cancer, ovarian cancer, testicular cancer, ependymoma, brain cancers such as high grade gliomas (HGG) including glioblastoma multiforme (GBM), neuroblastoma, and gastrointestinal (GI) cancers including appendiceal and colorectal cancer. A metastatic tumor can arise from a multitude of primary tumor types, including but not limited to those of prostate, colon, lung, breast, bone, and liver origin. Cancer can include malignancies of the various organ systems, such as cancers affecting the lung, breast, thyroid, lymphoid, gastrointestinal, or genito-urinary tract, as well as adenocarcinomas that include malignancies such as most colon cancers, renal-cell carcinoma, prostate cancer and/or testicular tumors, non-small cell carcinoma of the lung, cancer of the small intestine, and cancer of the esophagus. Other examples of cancers that can be treated include Hodgkin's and non-Hodgkin's lymphoma, rhabdosarcoma, Ewing's sarcoma, Wilm's tumor, and multiple myeloma.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, squamous cell carcinomas, and melanomas. Exemplary carcinomas include those forming from tissue of the cervix, lung, prostate, breast, head and neck, colon, and ovary. The term also includes carcinosarcomas, which include malignant tumors composed of carcinomatous and sarcomatous tissues. An adenocarcinoma refers to a carcinoma derived from glandular tissue or in which the tumor cells form recognizable glandular structures. Further details can be found in, for example, U.S. Pat. No. 7,834,147, which is incorporated herein by reference in its entirety.

In some embodiments, cancer includes breast cancer, e.g., metastatic triple negative breast cancer. The standard of care (SoC) for both unresectable locally advanced and metastatic TNBC (mTNBC represents both types of TNBC) have primarily been chemotherapies based on the National Comprehensive Cancer Network (NCCN) v1.2019 guidelines and the European School of Oncology-European Society for Medical Oncology (ESO-ESMO) 2018 guidelines for mTNBC treatment in China (National Comprehensive Cancer Network. NCCN clinical practice guidelines in oncology (NCCN guidelines): breast cancer (Version 1.2019). Accessed 8 Jul. 2019; Cardoso F, et al., Ann Oncol. 2018; 29:1634-57). Taxanes such as paclitaxel are recommended, unless contraindicated, as the first-line therapy for patients who have not previously received systemic anti-cancer treatment or who received neoadjuvant or adjuvant treatment more than one year ago with mTNBC recurrent, in this case, Taxanes can be reused as the first-line therapy (National Comprehensive Cancer Network. NCCN clinical practice guidelines in oncology (NCCN guidelines): breast cancer (Version 1.2019). Accessed 8 Jul. 2019; Cardoso F, et al., Ann Oncol. 2018; 29:1634-57). Nab-paclitaxel, a nanoparticle albumin-bound paclitaxel, demonstrated better efficacy and safety profiles in mTNBC treatment as compared to paclitaxel (Simon B. et al., Breast Cancer: Basic and Clinical Research, 2016, 10:25-36). The efficacy of paclitaxel/nab-paclitaxel monotherapy as the first-line therapy for mTNBC in a meta-analysis from 21,194 patients of 22 studies and other studies reported ORR: 23%-47.5%, PFS: 3.7-5.4 months, OS: 9.4-13.1 months among paclitaxel/nab-paclitaxel monotherapy with weekly dosing schedule (Awada A, et al., Ann Oncol. 2014; 25:824-31; Claire H. Li, et al. Breast Cancer Research (2019) 21:14327; H S Rugo, et al., J. Clin. One, 2015, 33(21):2361-2373.) In other embodiments, the cancer includes a lung cancer, a squamous cell lung cancer, a melanoma, a renal cancer, a liver cancer, a myeloma, a prostate cancer, a breast cancer, an ER+ breast cancer, an IM-TN breast cancer, a colorectal cancer, a colorectal cancer with high microsatellite instability, an EBV+ gastric cancer, a pancreatic cancer, a thyroid cancer, a nasopharyngeal cancer, (e.g., differentiated or undifferentiated metastatic or locally recurrent nasopharyngeal carcinoma), a hematological cancer, a non-Hodgkin lymphoma, a leukemia, and a metastatic lesion of the cancer. In some embodiments, the cancer is a triple negative breast cancer (TNBC) or gastric cancer.

As used herein, the terms “treat,” “treating,” “treatment” and variations thereof refer to partially or completely alleviating, inhibiting, ameliorating, or relieving the disease or condition (e.g., mTNBC or gastric cancer) from which the subject is suffering. “Treating cancer” means causing a partial or complete decrease in the rate of growth of a tumor, in the size of the tumor, in the rate of local or distant tumor metastasis, in the overall tumor burden in a subject, and/or any decrease in tumor survival, using the treatment methods described herein. In certain embodiments, “treat” and its variations refers to slowing the progression or reversing the progression of cancer (e.g., mTNBC) relative to an untreated control. The response to treatment using the disclosed methods can be evaluated by a number of parameters known in the art including those described below.

(1) The “objective response rate” or “ORR” is an important parameter to demonstrate the efficacy of a treatment in oncology (Aykan N F, Özath T. (2020) World J Clin Oncol. February 24; 11(2):53-73). It assesses the tumor burden (TB) after a given treatment in patients with solid tumors by determining the proportion of patients with tumor size reduction of a predefined amount and for a minimum time period. Tumors are evaluated using World Health Organization (WHO) criteria and Response Evaluation Criteria in Solid Tumors (RECIST), which are anatomic response criteria developed mainly for cytotoxic chemotherapy, and are based on anatomical imaging. ORR is measured as the total number of subjects whose best overall response (BOR) is either complete response (CR) or partial response (PR), divided by the total number of subjects in the population of interest.

(2) The “overall survival” or “OS” is the length of time from either the date of diagnosis or the start of treatment for a disease, such as cancer, that patients diagnosed with the disease are still alive. In the context of this disclosure, it is defined as the time from the date of randomization (the date of assigning a clinical trial subject to a particular treatment group) until date of death due to any cause.

(3) “Progression free survival” or “PFS” is the length of time during and after the treatment of a disease, such as cancer, that a patient lives with the disease but it does not get worse. In the context of this disclosure, it is defined as the time from the date of randomization (the date of assigning a clinical trial subject to a particular treatment group) to the date of disease progression or death.

(4) “Duration of Response” or “DoR” is the length of time that a tumor continues to respond to treatment without the cancer growing or spreading. In the context of this disclosure, it is defined as the time from documentation of a patient's positive response to treatment until disease progression.

(5) Disease control rate (DCR) is the percentage of patients with cancer who have achieved complete response (CR), partial response (PR), or stable disease (SD). In the context of this disclosure, it is defined as the proportion of participants whose best overall response (BOR) was complete response (CR), partial response (PR) and stable disease (SD) according to RECIST v1.1 (described, for example, in Eisenhauer et al., Eur J Cancer, (2009) 45(2):228-47, the disclosure of which is incorporated herein by reference in its entirety), or according to any subsequent update to that version of RECIST.

Thus, in some embodiments, treatment with the disclosed methods leads to an increase in at least one of the following parameters in the subject relative to a control population: (a) objective response rate (ORR); (b) overall survival (OS); (c) progression free survival (PFS); and (d) duration of response (DoR). For instance, treatment with the disclosed methods can increase any one or more of the ORR, OS, PFR, and/or DoR by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99%. In addition, treatment with the disclosed methods can increase the DCR in the treated population (i.e., the group of subjects treated with the disclosed methods) compared to the control population by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99%.

### Treatment Cycles

In some embodiments, the combination of the afuresertib, anti-PD-L1 antibody, and optional chemotherapeutic agent (e.g., nab-paclitaxel) of the disclosure can be administered to the subject for multiple treatment cycles (e.g., 1, 2, 3, 4, 5, 6, 7, or 8 cycles). The term “cycle” refers to a period of treatment followed by a period of rest (no treatment) that is repeated on a regular schedule. In some embodiments, in the combination therapy of the afuresertib, anti-PD-L1 antibody, and optional chemotherapeutic agent, one cycle is 3 weeks. In some embodiments, the subject is administered the afuresertib, anti-PD-L1 antibody, and chemotherapy (e.g., nab-paclitaxel) for 8 treatment cycles. Thereafter, the afuresertib and anti-PD-L1 antibody therapy continue as maintenance therapy per attending physician's discretion and/or standard of care. In some embodiments, the afuresertib and anti-PD-L1 antibody continue as maintenance therapy at the same doses as those administered during the first 8 treatment cycles. In some embodiments, the afuresertib and anti-PD-L1 antibody continue as maintenance therapy at a lower dose than that administered during the first 8 treatment cycles. In some embodiments, the afuresertib and anti-PD-L1 antibody maintenance therapy continues for an extended period of time, e.g., 12 months, 18 months, 24 months, 30 months, or longer. In some embodiments, the afuresertib and anti-PD-L1 antibody maintenance therapy continues for 12 months. In some embodiments, the afuresertib and anti-PD-L1 antibody maintenance therapy continues for 18 months. In some embodiments, the afuresertib and anti-PD-L1 antibody maintenance therapy continues for 24 months. In some embodiments, the afuresertib and anti-PD-L1 antibody maintenance therapy continues for 30 months.

The term “day 1 of the treatment cycle” or “day 1” refers to the first day that treatment with the afuresertib formulation commences in the subject.

In one example, Cycle 1 of treatment comprises the first three weeks (21 days+/−3 days) of treatment. The afuresertib formulation is administered once a day for 21 consecutive days. After administration of afuresertib, the anti-PD-L1 antibody is administered once on day 1. In some embodiments, a chemotherapeutic agent (e.g., nab-paclitaxel) is administered after afuresertib once on day 1 and once on day 8 of the treatment cycle.

In some embodiments, the afuresertib, or a pharmaceutically acceptable salt thereof, is administered orally. In some embodiments, the afuresertib, or a pharmaceutically acceptable salt thereof, is administered in a total daily dosage of from about 25 mg to about 200 mg; about 50 mg to about 200 mg; about 75 mg to about 150 mg; about 100 mg to about 125 mg; about 100 mg; about 110 mg; about 115 mg; about 120 mg; or about 125 mg on a free base basis. In some embodiments, the afuresertib or pharmaceutically acceptable salt thereof is administered to the patient in a dosage of about 100 mg, on a free base basis, once per day. In some embodiments, the afuresertib or pharmaceutically acceptable salt thereof is administered to the patient in a dosage of about 125 mg, on a free base basis, once per day.

In some embodiments, the anti-PD-L1 antibody is administered by intravenous infusion. In some embodiments, the infusion duration is 30 minutes. For example, the infusion duration is about 24 minutes, about 26 minutes, about 28 minutes, about 30 minutes, about 32 minutes, about 34 minutes, about 36 minutes, about 38 minutes, or about 40 minutes. In some embodiments, the anti-PD-L1 antibody is infused into the patient for 24 minutes. In some embodiments, the anti-PD-L1 antibody is infused into the patient for 26 minutes. In some embodiments, the anti-PD-L1 antibody is infused into the patient for 28 minutes. In some embodiments, the anti-PD-L1 antibody is infused into the patient for 30 minutes. In some embodiments, the the anti-PD-L1 antibody is infused into the patient for 32 minutes. In some embodiments, the anti-PD-L1 antibody is infused into the patient for 34 minutes. In some embodiments, the anti-PD-L1 antibody is infused into the patient for 36 minutes. In some embodiments, the anti-PD-L1 antibody is infused into the patient for 38 minutes. In some embodiments, the anti-PD-L1 antibody is infused into the patient for 40 minutes.

In some embodiments, the anti-PD-L1 antibody is administered to the subject once every three weeks at a dosage of about 500 mg to about 2000 mg; about 500 mg to about 1500 mg; about 800 mg to about 1200 mg; about 800 mg; about 900 mg; about 1000 mg; about 1100 mg; or about 1200 mg. In some embodiments, the anti-PD-L1 antibody is administered to the patient in a dosage of about 800 mg once every 3 weeks. In some embodiments, the anti-PD-L1 antibody is administered to the patient in a dosage of about 900 mg once every 3 weeks. In some embodiments, the anti-PD-L1 antibody is administered to the patient in a dosage of about 1000 mg once every 3 weeks. In some embodiments, the anti-PD-L1 antibody is administered to the patient in a dosage of about 1100 mg once every 3 weeks. In some embodiments, the anti-PD-L1 antibody is administered to the patient in a dosage of about 1200 mg once every 3 weeks.

In some embodiments, the chemotherapeutic agent (e.g., paclitaxel or nab-paclitaxel) is administered by intravenous infusion. In some embodiments, the chemotherapeutic agent is administered about 30 to about 60 minutes post administration of the anti-PD-L1 antibody. In some embodiments, the infusion duration is 30 minutes. For example, the infusion duration is about 24 minutes, about 26 minutes, about 28 minutes, about 30 minutes, about 32 minutes, about 34 minutes, about 36 minutes, about 38 minutes, or about 40 minutes. In some embodiments, the chemotherapeutic agent is infused into the patient for 24 minutes. In some embodiments, the chemotherapeutic agent is infused into the patient for 26 minutes. In some embodiments, the chemotherapeutic agent is infused into the patient for 28 minutes. In some embodiments, the chemotherapeutic agent is infused into the patient for 30 minutes. In some embodiments, the chemotherapeutic agent is infused into the patient for 32 minutes. In some embodiments, the chemotherapeutic agent is infused into the patient for 34 minutes. In some embodiments, the chemotherapeutic agent is infused into the patient for 36 minutes. In some embodiments, the chemotherapeutic agent is infused into the patient for 38 minutes. In some embodiments, the chemotherapeutic agent is infused into the patient for 40 minutes.

In some embodiments, the paclitaxel or nab-paclitaxel is administered to the subject on day 1 and day 8+/−1 day of a 3 weeks+/−3 days treatment cycle at a dosage of about 75 to about 200 mg/m² or at a dosage of about 125 mg/m². In some embodiments, the paclitaxel or nab-paclitaxel is administered to the subject on day 1 and day 8+/−1 day of a 3 weeks+/−3 days treatment cycle at a dosage of about 75 mg/m². In some embodiments, the paclitaxel or nab-paclitaxel is administered to the subject on day 1 and day 8+/−1 day of a 3 weeks+/−3 days treatment cycle at a dosage of about 100 mg/m². In some embodiments, the paclitaxel or nab-paclitaxel is administered to the subject on day 1 and day 8+/−1 day of a 3 weeks+/−3 days treatment cycle at a dosage of about 125 mg/m2.

In one aspect, the administration regimen per treatment cycle is as follows: (a) on day 1 of the treatment cycle: (i) administering afuresertib, or a pharmaceutically acceptable salt thereof; and (ii) after completion of step (a)(i), administering the anti-PD-L1 antibody; and (b) administering the afuresertib, or a pharmaceutically acceptable salt thereof, daily starting on day 2 of the treatment cycle. The duration of each treatment cycle is 3 weeks+/−3 days long.

In another aspect, the administration regimen per treatment cycle is as follows: (a) on day 1 of the treatment cycle: (i) administering about 25 to about 200 mg/kg of the afuresertib, or a pharmaceutically acceptable salt thereof, on a free base basis; and (ii) after completion of step (a)(i), administering about 500 to about 1500 mg of the anti-PD-L1 antibody; and (b) administering about 100 to about 125 mg/kg of the afuresertib, or a pharmaceutically acceptable salt thereof, on a free base basis daily starting on day 2 of the treatment cycle. The duration of each treatment cycle is 3 weeks+/−3 days.

In some embodiments of the above two aspects, the treatment further comprising administering paclitaxel or nab-paclitaxel on days 1 and 8 of the treatment cycle. In some embodiments, paclitaxel or nab-paclitaxel is administered after the administration of the anti-PD-L1 antibody on day 1 of the treatment cycle and after the administration of afuresertib, or a pharmaceutically acceptable salt thereof, on day 8 of the treatment cycle. In some embodiments, about 75 to about 125 mg/m² of paclitaxel or nab-paclitaxel is administered on day 1 of the treatment cycle. In some embodiments, about 30 minutes to about 60 minutes after administration of afuresertib, or a pharmaceutically acceptable salt thereof, about 75 to about 125 mg/m² of paclitaxel or nab-paclitaxel was administered on day 8 of the treatment cycle.

In some embodiments, the administration regimen per treatment cycle is as follows: (a) on day 1 of the treatment cycle: (i) orally administering about 100 to about 125 mg/kg of the afuresertib, or a pharmaceutically acceptable salt thereof, on a free base basis; (ii) after completion of step (a)(i), intravenously administering about 800 to about 1200 mg of the anti-PD-L1 antibody; and (iii) about 30 minutes to about 60 minutes after completion of step (a)(ii), intravenously administering about 125 mg/m² mg/kg of nab-paclitaxel; (b) orally administering about 100 to about 125 mg/kg of the afuresertib, or a pharmaceutically acceptable salt thereof, on a free base basis, on days 2-7 and 9-21 (+/−3 days) of the treatment cycle; and (c) on day 8 of the treatment cycle: (i) orally administering about 100 to about 125 mg/kg of the afuresertib, or a pharmaceutically acceptable salt thereof, on a free base basis; and (ii) after completion of step (c)(i), intravenously administering about 125 mg/m² of nab-paclitaxel; wherein each treatment cycle is 3 weeks+/−3 days long and the administration regimen repeats for at least 8 cycles.

As used herein, the term “simultaneous administration” refers to administration of a first treatment, such as administration of a first pharmaceutical composition (e.g., afuresertib or a pharmaceutically acceptable salt thereof), administration of a second treatment, such as administration of an anti-PD-L1 antibody), and administration of a third treatment, such as administration of a third pharmaceutical composition (e.g., chemotherapeutic agent such as nab-paclitaxel), wherein the first, second, and third treatments are separate and are administered at around the same time, i.e., the first, second, and third pharmaceutical compositions are administered within 30-60 minutes or within 2-6 hours of each other.

In some embodiments, the anti-PD-L1 antibody is administered to the subject orally, parenterally, subcutaneously, intravenously, rectally, intramuscularly, intraperitoneally, intranasally, transdermally, or by inhalation or intracavitary installation, topically, or by application to mucous membranes. In some embodiments, the anti-PD-L1 antibody is administered to the subject intravenously. In some embodiments, the afuresertib, or the pharmaceutically acceptable salt thereof, is administered to the subject orally. In some embodiments, the paclitaxel or nab-paclitaxel is administered to the subject intravenously. In some embodiments, the afuresertib, or the pharmaceutically acceptable salt thereof, and the anti-PD-L1 antibody are administered sequentially. In some embodiments, the afuresertib, or the pharmaceutically acceptable salt thereof, and the anti-PD-L1 antibody are administered simultaneously.

### Pharmaceutical Compositions and Formulations

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compounds of the disclosure or a pharmaceutically acceptable salt thereof, in combination with one or more pharmaceutically acceptable excipients. In making the compositions of the disclosure, the active ingredient is typically mixed with an excipient, diluted by an excipient or enclosed within such an excipient in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form. The term “unit dosage forms” refers to physically discrete units suitable as unitary dosages for human patients and other mammals, each unit containing a predetermined quantity of active material calculated to produce the desired therapeutic effect, in association with a suitable pharmaceutical excipient.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In some embodiments, afuresertib, or a pharmaceutically acceptable salt thereof, is formulated as part of a pharmaceutically acceptable composition further comprising one or more pharmaceutically acceptable excipients. In some embodiments, the pharmaceutical composition comprising afuresertib, or a pharmaceutically acceptable salt thereof, is suitable for oral administration. In some embodiments, the pharmaceutical composition comprising afuresertib or a pharmaceutically acceptable salt thereof, further comprises one or more of microcrystalline cellulose, mannitol, croscarmellose sodium and magnesium stearate. In some embodiments, the pharmaceutical composition comprising afuresertib or a pharmaceutically acceptable salt thereof is in the form of the following formulation:

TABLE 4
Afuresertib example formulation

| | Composition % |
|---|---|
| Component | by weight |
| Afuresertib HCl salt | 18.1 |
| Microcrystalline cellulose | 67.9 |
| Mannitol | 10.0 |
| Croscarmellose sodium | 2.8 |
| Magnesium stearate | 1.2 |
| Total unit dose | 100 |

In some embodiments, the pharmaceutical composition comprising afuresertib or a pharmaceutically acceptable salt thereof is in the form of the following formulation with the components in the following ranges:

TABLE 5
Afuresertib example formulation

| | Composition % |
|---|---|
| Component | range by weight |
| Afuresertib HCl salt | 15-20 |
| Microcrystalline cellulose | 65-70 |
| Mannitol | 5-15 |
| Croscarmellose sodium | 2.5-3.0 |
| Magnesium stearate | 1-2 |
| Total unit dose | 100 |

In some embodiments, the afuresertib is formulated as a tablet having the following composition:

TABLE 6
Afuresertib example formulation

| | Quantity mg/tablet | |
|---|---|---|
| | 50 mg Afuresertib | 75 mg Afuresertib |
| Component | (based on free base) | (based on free base) |
| Afuresertib HCl salt | 54.3 | 81.4 |
| Microcrystalline cellulose | 203.6 | 305.5 |
| Mannitol | 30.0 | 45.0 |
| Croscarmellose sodium | 8.3 | 12.5 |
| Magnesium stearate | 3.8 | 5.6 |
| Total unit dose | 300.0 | 450.0 |
| Opadry ® White aqueous | 6.0 to 12.0 | 9.0 to 18.0 |
| film coat | | |

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional excipients. In some embodiments, ointments can contain water and one or more hydrophobic excipients selected from, for example, liquid paraffin, polyoxyethylene alkyl ether, propylene glycol, white Vaseline, and the like. Excipient compositions of creams can be based on water in combination with glycerol and one or more other components, e.g. glycerinemonostearate, PEG-glycerinemonostearate and cetylsteaiyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication.

The amount of compound or composition administered to a patient varies depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous excipient prior to administration. The pH of the compound preparations typically is between 3 and 11, more preferably from 5 to 9 and most preferably from 7 to 8. It is understood that use of certain of the foregoing excipients, carriers, or stabilizers result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration.

The compositions of the disclosure can further include one or more additional pharmaceutical agents such as a chemotherapeutic, steroid, anti-inflammatory compound, or immunosuppressant.

In certain embodiments, the active compounds may be prepared with an excipient that protects the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

### Kits

The pharmaceutical compositions described herein may be included in a kit, pack, or dispenser, referred to collectively herein as a “kit,” optionally with instructions for administration of such pharmaceutical compositions. In some embodiments, a kit may include a first pharmaceutical composition comprising afuresertib (e.g., in solid form, such as a tablet), a second pharmaceutical composition comprising a PD-L1 antibody selected from atezolizumab, durvalumab, or avelumab, and a third pharmaceutical composition comprising a chemotherapeutic agent (e.g., nab-paclitaxel in a lyophilized form).

Optionally, additional pharmaceutical compositions also may be included in the kit. The kit also may include one or more containers of a pharmaceutically acceptable carrier (e.g., sterile water or saline) suitable for reconstituting or diluting each pharmaceutical composition included in the kit. The compositions and carrier(s) may be housed in vials or other suitable containers such as syringes. Typically, the first and second pharmaceutical compositions will be in separate containers in the kit. Each composition may be in the form of a dry powder, a liquid, a suspension in a liquid, frozen, or in any other suitable form.

It is to be understood that the disclosed methods and compositions may assume various alternative variations and step sequences, except where expressly specified to the contrary. Moreover, other than in any operating examples, or where otherwise indicated, all numbers such as those expressing values, amounts, percentages, ranges, subranges and fractions may be read as if prefaced by the word “about”, even if the term does not expressly appear. Accordingly, unless indicated to the contrary, the numerical parameters set forth in the specification and attached claims are approximations that may vary depending upon the results desired to be obtained by the disclosed methods. At the very least, and not as an attempt to limit the application of the doctrine of equivalents to the scope of the claims, each numerical parameter should at least be construed in light of the number of reported significant digits and by applying ordinary rounding techniques. Where a closed or open-ended numerical range is described herein, all numbers, values, amounts, percentages, subranges and fractions within or encompassed by the numerical range are to be considered as being specifically included in and belonging to the original disclosure of this application as if these numbers, values, amounts, percentages, subranges and fractions had been explicitly written out in their entirety.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

As used herein, unless indicated otherwise, a plural term can encompass its singular counterpart and vice versa, unless indicated otherwise. For example, although reference is made herein to “a” composition, a combination (i.e., a plurality) of these components can be used. In addition, in this application, the use of “or” means “and/or” unless specifically stated otherwise, even though “and/or” may be explicitly used in certain instances.

As used herein, “including”, “containing” and like terms are understood in the context of this application to be synonymous with “comprising” and are therefore open-ended and do not exclude the presence of additional undescribed and/or unrecited elements, materials, ingredients and/or method steps.

The compositions and methods are further supported by the information provided in the following Examples. It is to be understood that the embodiments described in the Examples are merely illustrative, and are not intended to limit the scope of the present disclosure, which will be limited only by the appended claims.

As used herein, the term “subject”, “individual” or “patient,” used interchangeably, includes human and non-human animals. The term includes, but is not limited to humans, mice, rats, other rodents, rabbits, dogs, cats, swine, cattle, sheep, horses, or primates. In some embodiments, the subject is a human subject, e.g., a human patient with cancer, e.g., a breast cancer such as metastatic triple negative breast cancer.

As used herein, “about” when referring to a measurable value such as an amount, a dosage, a temporal duration, and the like, is meant to encompass variations of ±10%. In certain embodiments, “about” can include variations of ±5%, ±1%, or +0.1% from the specified value and any variations there between, as such variations are appropriate to perform the disclosed methods.

All compounds, and pharmaceutically acceptable salts thereof, can be found together with other substances such as water and solvents (e.g., in the form of hydrates and solvates) or can be isolated. In some embodiments, the compounds of the disclosure, or salts thereof, are substantially isolated. By “substantially isolated” is meant that the compound is at least partially or substantially separated from the environment in which it was formed or detected. Partial separation can include, for example, a composition enriched in the compounds of the disclosure. Substantial separation can include compositions containing at least about 50%, at least about 60%, at least about 70%, at least about 80%, at least about 90%, at least about 95%, at least about 97%, or at least about 99% by weight of the compounds of the disclosure, or salt thereof. Methods for isolating compounds and their salts are routine in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the phrase “pharmaceutically acceptable excipient” refers to a pharmaceutically-acceptable material, composition, or vehicle, such as a liquid or solid filler, diluent, solvent, or encapsulating material. Excipients are generally safe, non-toxic and neither biologically nor otherwise undesirable and include excipients that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, “QD” is taken to mean a dosage administered to the patient once-daily. “Q3W” is taken to mean a dosage administered to the patient one every 3 weeks.

An “adverse event” (AE) is defined as any untoward medical occurrence in a clinical study patient administered a medicinal product which does not necessarily have a causal relationship with this treatment.

The following abbreviations are used herein:

AE=adverse event; CTCAE=Common Terminology Criteria for Adverse Events; DCR=disease control rate; DOR=duration of response; ECG=electrocardiogram; mCRPC=metastatic castration-resistant prostate cancer; ORR=overall response rate; OS=overall survival; PTEN=phosphatase and tensin homolog; RECIST 1.1=Response Evaluation Criteria in Solid Tumors version 1.1; rPFS=radiological progression free suivival.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Various modifications of the invention, in addition to those described herein, will be apparent to those skilled in the art from the foregoing description. Such modifications are also intended to fall within the scope of the appended claims. Each reference cited in the present disclosure, including all patent, patent applications, and publications, is incorporated herein by reference in its entirety.

### EXAMPLES

### Example 1: Afuresertib Suppresses Proliferation in Various Mouse Cell Lines Including MBT-2 Cells

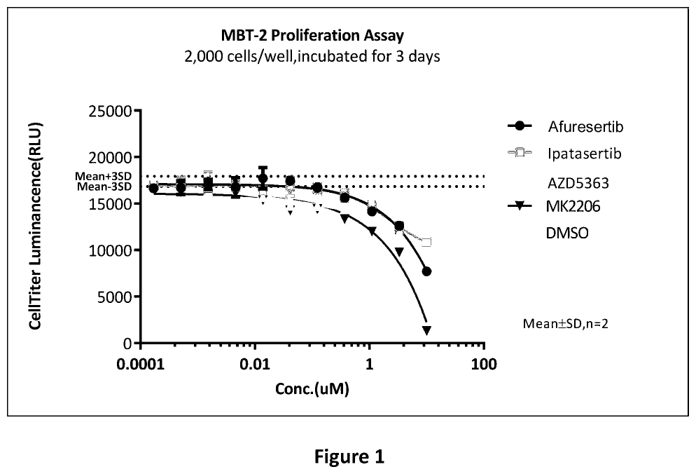

The objective of the study was to evaluate the ability of the AKT inhibitor afuresertib to suppress the proliferation of various mouse cancer cell lines, including a murine hepatoma cell line (Hepa 1-6), a murine mammary carcinoma cell line (EMT6), a murine prostate carcinoma cell line (RM-1), a murine pancreatic adenocarcinoma cell line (Pan02), a breast cancer cell line (4T1), a murine renal carcinoma cell line (Renca), a murine melanoma cell line (B16-F10), a murine melanoma cell line (LLC), a murine hepatocellular carcinoma cell line (H22), a murine colon adenocarcinoma cell line (MC38), a murine bladder tumor line-2 cell line (MBT-2), and a colon carcinoma cell line (CT26), and to select the appropriate cell line for further evaluation. The cell lines were obtained from Cobioer Biosciences Co. Ltd., Nanjing, China. 1,000 to 2,000 cells were seeded into 96-well plate, and induced with various concentrations of afuresertib (0.0001-10 μM) for 3 days at 37° C. with 5% CO₂. The number of viable cells in culture was determined based on quantitation of the amount of ATP following the manufacturer's instructions (CellTiter-Lumi Kit, Beyotime). Of these, the Mouse Bladder Tumor line-2 (MBT-2) was selected based on a favorable response profile to afuresertib. See FIG. 1.

### Example 2: MBT-2 Cells Express PD-L1

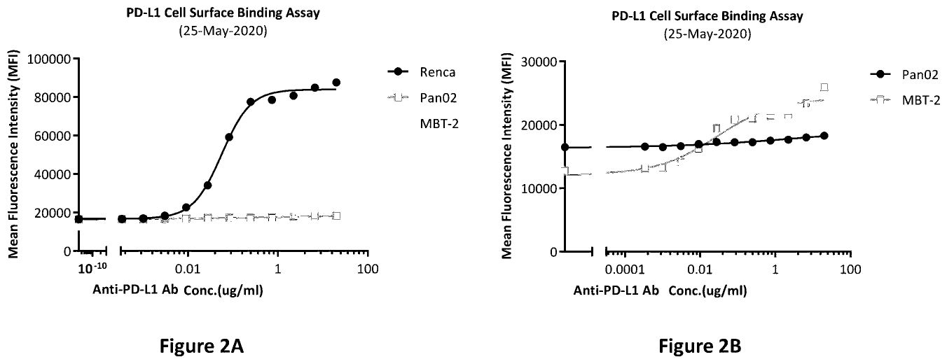

The objective of the study was to evaluate the expression of PD-L1 levels on various mouse cancer cell lines, including Hepa 1-6, EMT6, RM-1, Pan02, 4T1, Renca, B16-F10, LLC, H22, MC38, MBT-2, and CT26, and select the appropriate cell line for further evaluation. The cell lines were obtained from Cobioer Biosciences Co. Ltd., Nanjing, China. PD-L1 cell surface binding assays were evaluated by fluorescence associated cell sorting (FACS). Cells were stained in 3% FBS/PBS buffer with increasing concentration of anti-PD-L1 primary antibody for 1 h at 4° C. After the washing step, cells were incubated with anti-human APC-coupled secondary antibody (BioLegend) for 30 min at 4° C. in the dark for detection. The mean fluorescence intensity (MFI) increases in MBT-2 cells indicating that MBT2 is an appropriate PD-L1-expressing cell line to use. See FIGS. 2A-2B.

### Example 3: In Vivo Pharmacodynamic Study of Test Compounds in MBT-2 Syngeneic Subcutaneous Transplanted Tumor Model of C3H Mice

Based on the in vitro studies shown in Examples 1 and 2, Mouse Bladder Tumor line-2 (MBT-2) was selected as being afuresertib-responsive and PD-L1-expressing. The aim of the present study was to evaluate the in vivo antitumor effect of single use or the combined use of the test compounds Afuresertib, anti-PD-L1 Ab, or an Isotype, using MBT-2 syngeneic subcutaneous transplanted tumor model of C3H mice.

6-8 weeks old C3H female mice (Beijing Vital River Laboratory Animal Technology Co., Ltd.) weighing 18-20 g were housed in the experimental environment for 3-7 days before experiment initiation. The animals were housed in an SPF-grade animal room with IVCs (individually ventilated caging system) (4 animals per cage). The animal information tags of each cage indicated quantity, sex, strain, date of receipt, dosing regimen, study number, group number, and study initiation date. All the cages, bedding materials, and drinking water were sterilized prior to use.

Anti-PD-L1 antibody (Laekna Therapeutics Shanghai Co., Ltd;) was stored as a solution at a concentration of 5.03 mg/mL at −20° C. Human IgG4 isotype (Laekna Therapeutics Shanghai Co., Ltd;) was stored as a solution at a concentration of 12.05 mg/mL at −20° C.

The study design is shown below in Table 7.

TABLE 7
Study Design

| | | | | Dosing | | |
|---|---|---|---|---|---|---|
| Group | | Treatment | Dose | volume | Route of | Dosing |
| no. | N^{a} | regimen | (mg/kg) | (μL/g) | administration | frequency* |
| 1 | 8 | Vehicle + | 10 | 10 | PO + IP | (QD + BIW)*3 |
| | | Isotype | (Isotype) | | | weeks |
| 2 | 8 | Afuresertib + | 75 + 10 | 10 | PO + IP | (QD + BIW)*3 |
| | | Isotype | | | | weeks |
| 3 | 8 | Anti-PD-L1 + | 10 | 10 | IP + PO | (BIW + QD)*3 |
| | | Vehicle | (anti-PD-L1) | | | weeks |
| 4 | 8 | Afuresertib + | 75 +10 | 10 | PO + IP | (QD + BIW)*3 |
| | | anti-PD-L1 | | | | weeks |

Note:
^{a}N: Number of mice per group; PO: Oral; IP: Intraperitoneal; QD: daily; BIW: twice a week
*After 3-week administration, the animals in Groups 2-4 were further observed until D 45, followed by a survival analysis

### Cell Culture

MBT-2 cells were cultured in EMEM medium with 10% fetal bovine serum (FBS), 100 U/mL penicillin, and 100 μg/mL streptomycin at 37° C. and 5% CO₂. They were routinely subcultured twice a week. When the cells remained in the exponential phase, they were harvested, counted, and inoculated.

### Inoculation of Tumor Cells

5×10⁵ MBT-2 cells (0.1 mL) were subcutaneously inoculated into the right back of neck of each mouse. Administration by different groups was started when the mean tumor volume was about 73 mm³.

### Preparation of Test Articles

Test articles were prepared as shown in Table 8 below.

TABLE 8
Preparation of Test Articles

| | Purity | Salt | Concentration | | Storage |
|---|---|---|---|---|---|
| Test article | (%) | coefficient | (mg/mL) | Preparation method | condition |
| Vehicle | — | — | — | 1% w/v methylcelluose: Weigh 10 g | Normal |
| | | | | methylcelluose and add 600 mL | temperature |
| | | | | double distilled water, stir well, | |
| | | | | and add double distilled water to | |
| | | | | a constant volume of 1000 mL | |
| Isotype | >98 | — | 1.0 | Pipet 0.34-mL 12.05 mg/mL | — |
| | | | | Isotype and dissolve in 3.67 mL | |
| | | | | PBS | |
| Afuresertib | 98 | 1.0854 | 7.5 | Weigh 124.60 mg Afuresertib and | 4° C. |
| | | | | dissolve in 15.00 mL vehicle | |
| Anti-PD-L1 Ab | >98 | — | 1.0 | Pipet 0.81-mL 5.03 mg/mL | — |
| | | | | anti-PD-L1 Ab and dissolve in | |
| | | | | 3.19 mL PBS | |

Note:
Afuresertib was prepared once every 3 days, while Isotype and anti-PD-L1 Ab were dispensed in advance and freshly prepared prior to use.

### Daily Observation of Laboratory Animals

The development of and any amendment to this study protocol were assessed and approved by the Institutional Animal Care and Use Committee (IACUC) of WuXi AppTec (Shanghai) Co., Ltd. The use of the laboratory animals and welfare were implemented in accordance with relevant regulations of the Association for Assessment and Accreditation of Laboratory Animal Care International (AAALAC).

### Tumor Measurement and Study Indicators

Study indicators were used to investigate whether the tumor growth was suppressed, retarded or cured. A vernier caliper was used to measure the tumor diameter 3 times a week. The tumor volume was calculated using the following formula: V=0.5a×b², where a and b respectively representing the major diameter and the minor diameter of a tumor.

The tumor suppression efficacy of the compounds was evaluated with TGI (%) or relative tumor proliferation rate (T/C, %). TGI (%) reflects the tumor growth inhibition rate. Calculation of TGI (%): TGI (%)=[(1−(mean tumor volume in a treatment group at the end of administration−mean tumor volume in the treatment group at the start of administration)/(mean tumor volume in the vehicle control group at the end of treatment−mean tumor volume in the vehicle control group at the start of treatment)]×100. T/C (%)=mean tumor volume in a treatment group at the end of administration/mean tumor volume in the vehicle control group at the end of treatment×100.

On PG-D24, mice A37530 and A37519 in group 2 were euthanized, then their tumors were collected and weighed, from which paraffin sections were made.

### Statistical Analysis

Statistical analysis included the mean value and standard error of mean (SEM) of tumor volumes in each group at each time point. Comparison between the treatment groups and vehicle control group was analyzed using one-tailed T-Test, and all the data were analyzed using GraphPad Prism 5.0. P<0.05 was considered as a significant difference.

### Study Results

### (a) Changes in Weight

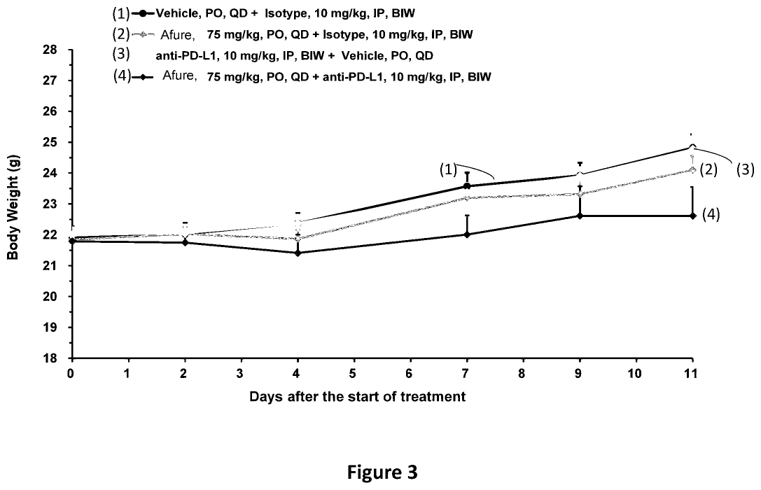

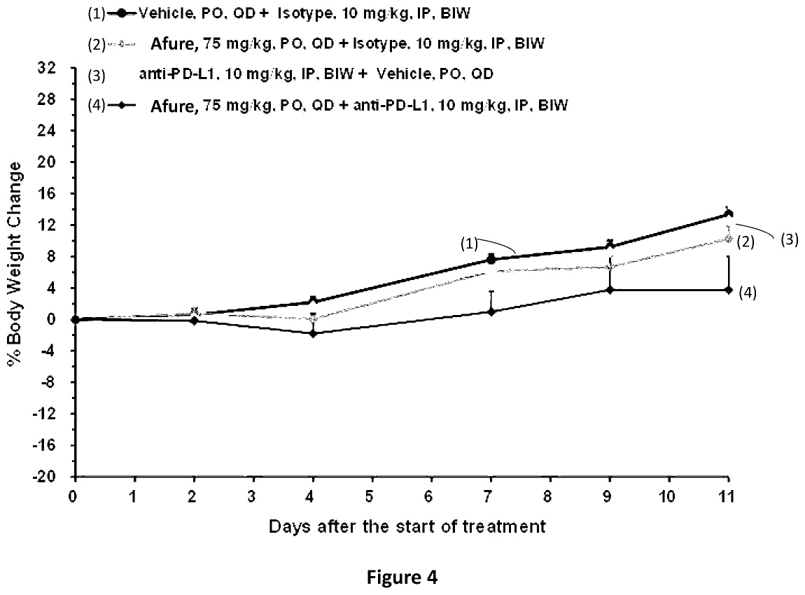

During the study, due to notable weight loss of one mouse in the treatment groups, the drugs were discontinued on PG-D5 until study completion. None of the other mice experienced notable weight loss. The data are shown in FIGS. 3 and 4.

### (b) Tumor Volumes

Tumor volume changes in MBT-2 tumor-bearing mice of each group after treatment with Isotype, Afuresertib or anti-PD-L1 Ab are shown in Table 9.

TABLE 9
Tumor volumes per group at different time points (mm³)^{a}

| | Compound | Days of administration | | | | | |
|---|---|---|---|---|---|---|---|
| Group no. | treatment | 0 | 2 | 4 | 7 | 9 | 11 |
| 1 | Vehicle, PO, QD + | 73 ± 8 | 164 ± 27 | 346 ± 45 | 910 ± 107 | 1567 ± 195 | 2173 ± 261 |
| | Isotype, 10 mg/kg, | | | | | | |
| | IP, BIW | | | | | | |
| 2 | Afuresertib, 75 mg/kg, | 73 ± 8 | 143 ± 27 | 319 ± 76 | 755 ± 170 | 1249 ± 298 | 1708 ± 371 |
| | PO, QD + | | | | | | |
| | Isotype, 10 mg/kg, | | | | | | |
| | IP, BIW | | | | | | |
| 3 | Anti-PD-L1, 10 mg/kg, | 73 ± 8 | 154 ± 32 | 293 ± 64 | 596 ± 188 | 1027 ± 325 | 1428 ± 531 |
| | IP, BIW + | | | | | | |
| | Vehicle, PO, QD | | | | | | |
| 4 | Afuresertib, 75 mg/kg, | 73 ± 8 | 134 ± 25 | 277 ± 76 | 529 ± 199 | 789 ± 307 | 946 ± 379 |
| | PO, QD + | | | | | | |
| | Anti-PD-L1, 10 mg/kg, | | | | | | |
| | IP, BIW | | | | | | |

Note:
^{a}mean ± SEM

### (c) Tumor Growth Curves

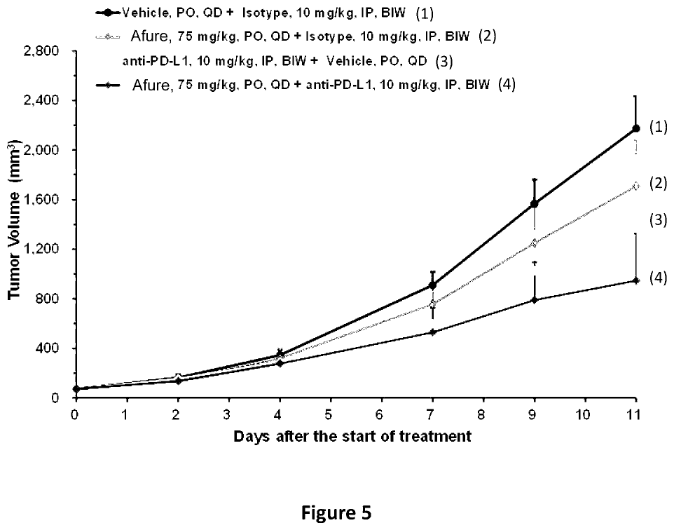

Tumor growth curves are shown in FIG. 5.

### (d) Antitumor Efficacy Evaluation Indicators

TABLE 4
Antitumor efficacy evaluation of test articles on MBT-2
subcutaneous transplanted tumor model (calculated based
on the tumor volume on day 11 post administration)

| | | Tumor | | | P value^{b} | P value^{b} |
|---|---|---|---|---|---|---|
| Group | Compound | volume | T/C | TGI | (vs. Group | (vs. Group |
| no. | treatment | (mm³)^{a} | (%) | (%) | 1) | 3) |
| 1 | Vehicle, PO, QD + | 2173 ± 261 | — | — | — | — |
| | Isotype, 10 mg/kg, | | | | | |
| | IP, BIW | | | | | |
| 2 | Afuresertib, 75 mg/kg, | 1708 ± 371 | 78.6 | 22.1 | P > 0.05 | — |
| | PO, QD + Isotype, | | | | | |
| | 10 mg/kg, IP, BIW | | | | | |
| 3 | Anti-PD-L1, 10 mg/kg, | 1428 ± 531 | 65.7 | 35.5 | P > 0.05 | — |
| | IP, BIW + Vehicle, | | | | | |
| | PO, QD | | | | | |
| 4 | Afuresertib, 75 mg/kg, | 946 ± 379 | 43.5 | 58.4 | P < 0.01 | P > 0.05 |
| | PO, QD + Anti-PD-L1, | | | | | |
| | 10 mg/kg, IP, BIW | | | | | |

Notes:
^{a}mean ± SEM
^{b}P values for each treatment group and the vehicle control group were calculated using one-tailed T Test.

### (d) Survival Curves

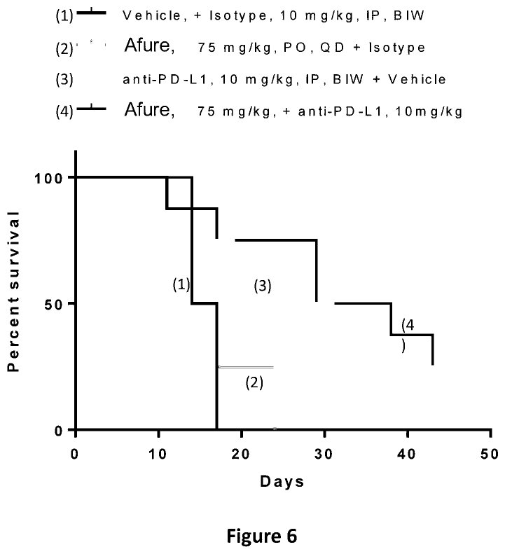

Survival curves of the animals in various groups are shown in FIG. 6.

### Statistical Analysis of Survival Curves

TABLE 10
Statistical analysis of survival time in each treatment group

| | | Median | P value^{a} |
|---|---|---|---|
| Group | | survival | (vs. |
| no. | Treatment regimen | (days) | Group 1) |
| 1 | Vehicle, PO, QD + Isotype, | 15.5 | — |
| | 10 mg/kg, IP, BIW | | |
| 2 | Afuresertib, 75 mg/kg, PO, | 15.5 | P > 0.05 |
| | QD + Isotype, 10 mg/kg, | | |
| | IP, BIW | | |
| 3 | Anti-PD-L1, 10 mg/kg, IP, | 27.5 | P < 0.05 |
| | BIW + Vehicle, PO, QD | | |
| 4 | Afuresertib, 75 mg/kg, | 33.5 | P < 0.01 |
| | PO, QD + Anti-PD-L1, | | |
| | 10 mg/kg, IP, BIW | | |

Note:
^{a}P values for each treatment group and the vehicle control group were calculated using Log-rank (Mantel-Cox) test.

### Results and Discussion

In this study, the in vivo efficacy of Isotype, Afuresertib, or anti-PD-L1 was evaluated in a Mouse Bladder Tumor line-2 (MBT-2) subcutaneous transplanted mouse tumor model. Tumor volumes of various test groups at different time points are shown in Tables 3 and 4 as well as FIG. 5.

After administration for 11 consecutive days, the mean tumor volume of tumor-bearing mice in the control group (vehicle+10 mg/kg Isotype) was 2173 mm³. Compared with the results in the control group, 75 mg/kg Afuresertib plus 10 mg/kg Isotype (TV=1708 mm³, TGI=22.1%, P>0.05) and 10 mg/kg anti-PD-L1 Ab plus vehicle (TV=1428 mm³, TGI=35.5%, P>0.05) did not show notable inhibition on the growth of MBT-2 tumors. Combination treatment with 75 mg/kg Afuresertib and 10 mg/kg anti-PD-L1 showed notable inhibition on the growth of MBT-2 tumors, with statistical differences compared with the control group (TV=946 mm³, TGI=28.4%, P<0.05).

Effects of various treatment groups on the changes in weights of tumor-bearing mice are shown in FIGS. 3 and 4. During the study, due to notable weight loss of the one mouse in the treatment groups, the drugs were discontinued on PG-D5. None of the other mice experienced notable weight loss.

### Conclusion

These results show that the combination of Afuresertib plus anti-PD-L1 antibody is effective in inhibiting the growth of MBT-2 tumors in mice and enhancing survival of the animals.

### Example 4: Dose Escalation and Efficacy Study of Anti-PD-L1 Antibody+Afuresertib+Nab-Paclitaxel in Advanced Solid Tumors

A clinical trial was designed to evaluate the dosage regimen and efficacy of the combination of an anti-PD-L1 antibody and Afuresertib, further in combination with nab-paclitaxel.

### Overall Design

The Phase I part of the study is a dose-escalation study to evaluate the safety, tolerability, and determine the maximum tolerated dose (MTD) and recommended Phase II dose (RP2D) of anti-PD-L1 antibody, afuresertib and nab-paclitaxel as a combination treatment in patients with advanced solid tumors (including metastatic triple negative breast cancer; mTNBC). The secondary objective of the Phase I part of the study assesses the preliminary anti-tumor activity of the anti-PD-L1 antibody, afuresertib and nab-paclitaxel; characterizes the immunogenicity of the anti-PD-L1 antibody; and characterizes the pharmacokinetics (PK) of the anti-PD-L1 antibody and afuresertib in patients receiving combination treatment of the anti-PD-L1 antibody and afuresertib and nab-paclitaxel.

The Phase II part of the study assesses the anti-tumor activity (based on ORR) of the anti-PD-L1 antibody, afuresertib and nab-paclitaxel in patients with mTNBC who have not received any systemic anti-tumor treatment or received neoadjuvant or adjuvant therapy more than one year before study enrollment. The secondary objective of the Phase II part of the study assesses the clinical efficacy of the triple combination therapy of the anti-PD-L1 antibody, afuresertib and nab-paclitaxel, as well as the combinations of the anti-PD-L1 antibody plus nab-paclitaxel and afuresertib plus nab-paclitaxel and nab-paclitaxel monotherapy in patients with TNBC. Further, a secondary objective is to evaluate the safety and tolerability of the triple combination therapy of the anti-PD-L1 antibody and afuresertib and nab-paclitaxel, as well as the combinations of anti-PD-L1 antibody plus nab-paclitaxel and afuresertib plus nab-paclitaxel, and to characterize the immunogenicity of the anti-PD-L1 antibody.

In Phase II, additional exploratory objectives include assessing the clinical efficacy (based on iRECIST) of the triple combination therapy of the anti-PD-L1 antibody and afuresertib and nab-paclitaxel, as well as the combination of the anti-PD-L1 antibody plus nab-paclitaxel in patients with mTNBC; exploring the relationship between PTEN/PI3K/AKT, BRCA alternations and anti-tumor activity and safety of each combination treatment; and exploring the relationship between baseline PD-L1 levels and anti-tumor activity and safety of each combination treatment.

The following abbreviations are used in the study: AE=adverse event; CTCAE=common terminology criteria for adverse events; DCR=disease control rate; DOR=duration of response; ECG=electrocardiogram; ITT=intent to treat; ORR=overall response rate; OS=overall survival; PTEN=phosphatase and tensin homolog; RECIST 1.1=Response Evaluation Criteria in Solid Tumors version 1.1.

### Trial Design

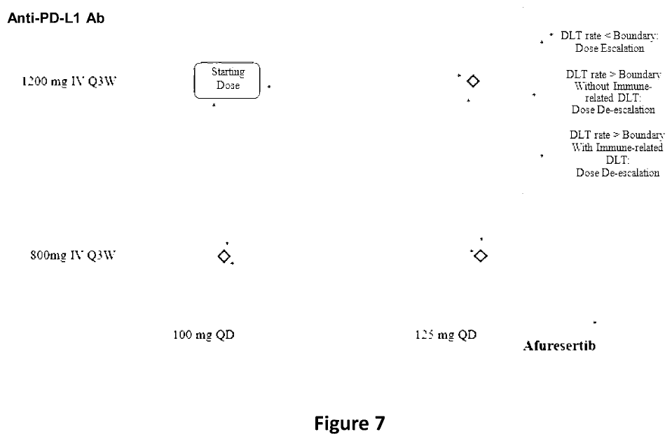

The starting dose of the triple combination in the phase I dose escalation is as follows: anti-PD-L1 antibody 1200 mg IV Q3W+afuresertib 100 mg QD+nab-paclitaxel 125 mg/m² IV D1, D8 Q3W. The dose escalation in afuresertib starts from 100 mg QD to 125 mg QD. The dose of the anti-PD-L1 antibody and nab-paclitaxel dose remains unchanged unless immune-related dose-limiting toxicity (DLT) is observed. In such cases, the anti-PD-L1 Ab dose is de-escalated to 800 mg IV Q3W, while afuresertib and nab-paclitaxel remains at the corresponding dose levels. If nab-paclitaxel 125 mg/m² IV D1, D8 Q3W is not tolerated, the dose may be de-escalated to 100 mg/m² IV D1, D8 Q3W. The dose escalation scheme is shown in FIG. 7. IV=intravenous; Q3W=every 3 weeks; QD=once daily; D1=day 1; D8=day 8.

The Phase I dose escalation for triple combination therapy with BOINcomb design has the following provisional dose levels of anti-PD-L1 antibody and afuresertib in the dose escalation of the triple combination therapy:
anti-PD-L1 antibody: 1200 mg IV Q3W, 800 mg IV Q3W
Afuresertib: 100 mg QD, 125 mg QD

The starting dose level of the triple combination therapy is −anti-PD-L1 antibody 1200 mg IV Q3W+Afuresertib 100 mg QD+nab-paclitaxel 125 mg/m² IV D1, D8 Q3W.

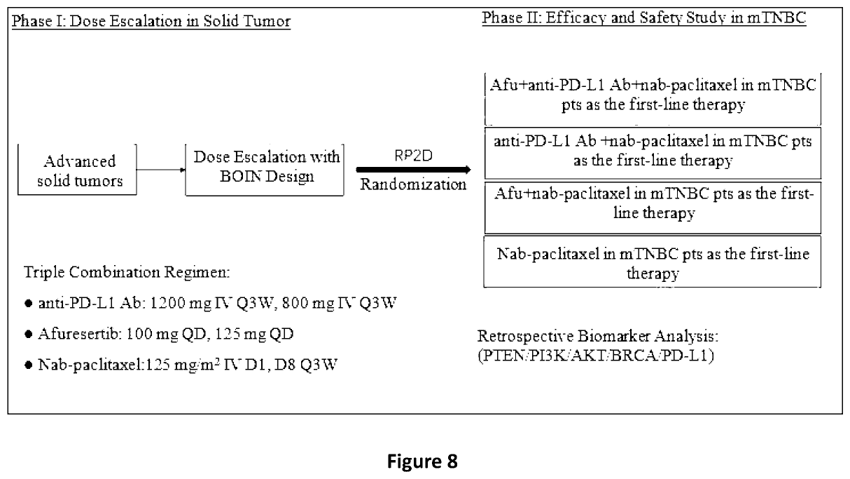

The safety (including DLT), tolerability, and PK are the study endpoints of phase I. Phase II is a multi-center, randomized, open-label, four parallel arms designed study to assess the anti-cancer efficacy, safety and biomarker correlation to efficacy. The therapies in the four treatment arms are: anti-PD-L1 antibody+afuresertib+nab-paclitaxel; anti-PD-L1 antibody+nab-paclitaxel; afuresertib+nab-paclitaxel; and nab-paclitaxel monotherapy, respectively. The ORRs based on RECIST 1.1 of four therapies are the primary endpoint, whereas the DOR, PFS, DCR are the secondary endpoints in phase II study. The study design is shown in FIG. 8.

The dose escalation part of the study employs the Bayesian optimal interval for Drug Combination Trials (BOINcomb) design (Liu S. and Yuan Y. (2015) Journal of the Royal Statistical Society: Series C, 64, 507-523.; Lin R. and Yin, G. (2015). Statistical Methods in Medical Research, DOI: 10.1177/0962280215594494; Yuan, Y. and Zhang, L. (2017) Designing Early-Phase Drug Combination Trials. Handbook of Methods for Designing, Monitoring, and Analyzing Dose Finding Trials, edited by O'Quigley J., Iasonos, A and Bornkamp, B., Chapter 6, p 109-p 126) to determine the MTD. The target toxicity rate for the MTD is 0.3 and the maximum sample size is 21. MTD is defined as the combination dose level with a DLT rate that is closest to the target toxicity rate (0.3). Patients are enrolled in cohorts of size 3. DLTs are defined in the DLT Section below, and only those DLTs that occur within the first cycle are used for dose finding.

RP2D is determined after the integrated data, including safety, PK/PD and efficacy, are reviewed by Safety Review Committee (SRC). If necessary, additional dose escalation schedule may be explored. Once the RP2D of combination therapy with the anti-PD-L1 antibody and afuresertib and nab-paclitaxel has been established, 80 mTNBC patients who meet inclusion and exclusion criteria are randomized with a ratio of 1:1:1:1 to each of the four treatment arms to evaluate the efficacy and safety of each combination therapy and the monotherapy. Patients with mTNBC have not received any systemic anti-cancer treatment or received only neoadjuvant or adjuvant treatment over 1 year from the study randomization date. The ORR as the primary endpoint is estimated for individual arms to demonstrate the preliminary anti-tumor activity of the combination therapies in this patient population. Tumor biomarkers, including PD-L1 level and PI3K/PTEN/AKT/BRCA alterations are tested retrospectively.

In the Phase II stage, an ORR around 70% is expected by the triple combination therapy with the anti-PD-L1 Ab and afuresertib and nab-paclitaxel as compared to the historical control of paclitaxel monotherapy, the current first-line therapy of mTNBC in China. The ORRs with a range of 23%-47.5% were reported from 21,194 mTNBC patients in 22 studies under paclitaxel/nab-paclitaxel monotherapy (Peter Schmid, et al., N Engl J Med. 2018 Nov. 29; 379(22):2108-2121; A Awada, et al. Ann Oncol. 2014 April; 25(4):824-831; Claire H Li, et al. Breast Cancer Res. 2019 Dec. 16; 21(1):143; Hope S. Rugo, et al. CALGB 40502/NCCTG N063H (Alliance). J Clin Oncol. 2015 Jul. 20; 33(21): 2361-2369; Sylvia Adams, et al. JAMA Oncology 2018; 5(3); 5152). The ORR of 34% with nab-paclitaxel monotherapy from a phase III study in 521 patients with mTNBC without testing PD-L1 status (Hope S. Rugo, et al. CALGB 40502/NCCTG N063H (Alliance). J Clin Oncol. 2015 Jul. 20; 33(21): 2361-2369), whereas the ORR of 32% treated with paclitaxel monotherapy from LOTUS Study can be used as the historical control for mTNBC patients with PD-L1 negative status (Kim S B, Dent R. Im S A. et al., Lancet Oncology, 2017, 18(10):1360-1372). The above ORRs of 38% and 32% with paclitaxel monotherapy are selected as the historical control because these ORRs came from two phase III studies with a large sample size, same paclitaxel dosing regimen and similar mTNBC patient population (first-line therapy) compared to the current phase I study. Whereas the ORR ranged from 23% to 47.5% came from a mixed mTNBC population who failed one to three lines of therapy and mixed treatment with either paclitaxel or nab-paclitaxel among these studies (Peter Schmid, et al., N Engl J Med. 2018 Nov. 29; 379(22):2108-2121; A Awada, et al. Ann Oncol. 2014 April; 25(4):824-831; Claire H Li, et al. Breast Cancer Res. 2019 Dec. 16; 21(1):143; Hope S. Rugo, et al. CALGB 40502/NCCTG N063H (Alliance). J Clin Oncol. 2015 Jul. 20; 33(21): 2361-2369; Sylvia Adams, et al. JAMA Oncology 2018; 5(3); 5152). Therefore, the ORR change from 38% to 70%, is considered as a clinically meaningful improvement in mTNBC patients. The two double combinations provide not only additional treatment options, especially in certain biomarker defined patient populations such as PD-L1 negative or PTEN loss, but also allow us to have a better understanding of the individual contributions to the overall efficacy and safety of the combination therapy. This study explores the preliminary efficacy of each combination therapy in mTNBC as first line treatment by estimation of ORR in each arm.

### Pharmacokinetic Sampling

In Phase I, blood sampling is performed in 3-6 patients for the anti-PD-L1 antibody and afuresertib PK study. The PK time points are listed in Tables 6 and 7. The pre-dose sample on Day 1 is collected within 1 hour before dosing; for other time points, samples can be obtained ±5 minutes of the scheduled sampling time for samples to be collected ≤1 hour after a dose, and ±10 minutes of the scheduled sampling time for samples to be collected >1 and ≤24 hours after a dose.

Determination of plasma concentrations of afuresertib and the anti-PD-L1 antibody is performed by a qualified lab using a validated analytical method. Detailed sample collection, label, storage, and shipment is described in the Laboratory Manual. Afuresertib and the anti-PD-L1 antibody concentration data is used to calculate PK parameters.

TABLE 11
The anti-PD-L1 Ab PK Sampling Phase I

| Cycle* | Day | Scheduled Time Point (h) |
|---|---|---|
| 1 | 1 | Pre-infusion of Cycle 1 |
| 1 | 1 | 1 h post-infusion (±5 min) # |
| 1 | 2 | 24 h post-infusion (±2 h) |
| 1 | 8 | 168 h post-infusion (±8 h) |
| 1 | 15 | 336 h post-infusion (±24 h) |
| 2 | 1 | Pre-infusion of Cycle 2 |
| 3 | 1 | Pre-infusion of Cycle 3 |
| 3 | 1 | 1 h post-infusion (±5 min) # |
| 3 | 2 | 24 h post-infusion (±2 h) |
| 3 | 8 | 168 h post-infusion (±8 h) |
| 3 | 15 | 336 h post-infusion (±24 h) |
| 4 | 1 | Pre-infusion of Cycle 4 |
| 6 | 1 | Pre-infusion of Cycle 6 |
| 8 | 1 | Pre-infusion of Cycle 8 |
| Unscheduled | | Anytime |

*Note:
“Cycle” refers to the anti-PD-L1 Ab dosing cycle, with three weeks as a cycle.
# Note:
After completion of the anti-PD-L1 Ab dosing.

TABLE 12
Afuresertib PK Sampling Phase I

| Cycle | Day | Scheduled Time Point (h) |
|---|---|---|
| 1 | 1 | Pre-dosing of Cycle 1 and 0.5, |
| | | 1, 2, 4, 6, 8 h post-dosing |
| 1 | 2 | 24 h post-dosing (±2 h) |
| 1 | 15 | Pre-dosing and 0.5, 1, 2, 4, |
| | | 6, 8 h post-dosing |
| 1 | 16 | 24 h post-dosing (±2 h) |
| 2 | 15 | Pre-dosing of Cycle 2 |
| | | (steady-state, trough) |
| 3 | 15 | Pre-dosing of Cycle 3 |
| | | (steady-state, trough) |
| Unscheduled | | Anytime |

Patients are discharged after completion of the 8-hour time point and come back to the office for the 24-hour pharmacokinetic sampling.
Blood samples can be obtained ±5 minutes of the scheduled sampling time for samples to be collected ≤1 hour after a dose, and ±10 minutes of the scheduled sampling time for samples to be collected >1 and ≤24 hours after a dose.
After completion of the 8-hour time point, patients may stay in the office or are discharged and come back to the office for the 24-hour pharmacokinetic sampling.
Samples can be obtained ±5 minutes of the scheduled sampling time for samples to be collected ≤1 hour after a dose, and ±10 minutes of the scheduled sampling time for samples to be collected >1 and ≤24 hours after a dose.

### End of Study

The main data analysis and reporting is performed when all patients have completed treatment until disease progression, dropped out from the study (due to reasons as ICF withdrawal, investigator's decision or non-compliance), or died for any reason.

The end of study for Phase II is defined as the time when 90% of patients in the Phase II part have progressed, dropped out from the study (due to reasons as ICF withdrawal, investigator's decision or non-compliance), or died for any reason.

After study completion, patients who discontinue nab-paclitaxel due to intolerance and continue to experience clinical benefit may continue study treatment with the anti-PD-L1 antibody plus afuresertib until disease progression, death, unacceptable toxicity, or start new anticancer treatment. All AEs, SAEs, study drug dosing and dose reduction of treatment are collected, however, other standard procedures and tests needed to treat and evaluate patients is not routinely collected and monitored by sponsor.

### Afuresertib

Two (2) strengths of afuresertib are supplied for oral administration equivalent to 50 mg and 75 mg of the free base. The drug product is supplied as immediate release tablets intended for oral administration. The tablets are packaged in white high-density polyethylene (HDPE) bottles with white plastic, induction-seal, child-resistant caps. The tablet bottles may contain desiccant. The recommended storage conditions, and expiry date where required, are stated on the product label. Afuresertib is to be stored between 10° C. and 30° C. in the provided bottle.

### Anti-PD-L1 Antibody

anti-PD-L1 antibody concentrate for solution for infusion is a clear and colorless solution in glass vial with a gray rubber stopper which is sealed with an aluminum cap with plastic flip-off disk. Each vial of anti-PD-L1 antibody concentrate contains 100 mg/1 mL anti-PD-L1 antibody monoclonal antibody. The recommended storage conditions, and expiry date where required, are stated on the product label.

### Nab-Paclitaxel

Nab-paclitaxel is a white to pale yellow sterile lyophilized cake or powder. Prior to reconstitution, nab-paclitaxel is a lyophilized cake or powder intended for reconstitution with 0.9% sodium chloride injection prior to IV infusion. Nab-paclitaxel for IV administration is centrally supplied by the sponsor in the commercial package with the required regulatory information. The recommended storage conditions and expiry date for nab-paclitaxel for injection are stated on the product label.

### Treatment of Patients

This is a multi-center, open-label, dose-escalation and proof-of-concept Phase I/II study to assess RP2D, safety, tolerability and anti-tumor activity of afuresertib and the anti-PD-L1 Ab and nab-paclitaxel administered as a combination treatment. This trial is a two-phase design for exploratory and proof of concept purpose.

The dose levels of the anti-PD-L1 Ab and afuresertib, and the dose levels of the triple combination therapy (the anti-PD-L1 Ab, afuresertib and nab-paclitaxel) are described earlier in this section. The starting dose of the triple combination in the phase I dose escalation is the anti-PD-L1 Ab 1200 mg IV Q3W+afuresertib 100 mg QD+nab-paclitaxel 125 mg/m² IV D1, D8 Q3W. The afuresertib dose is escalated from 100 mg QD to 125 mg QD if DLTs are below the boundary. The dose of the anti-PD-L1 Ab and nab-paclitaxel dose remains unchanged unless immune-related or nab-paclitaxel-related DLT is observed. If immune-related DLT is observed, the anti-PD-L1 Ab dose is be de-escalated to 800 mg Q3W. If nab-paclitaxel 125 mg/m² IV D1, D8 Q3W is not tolerated, the dose is de-escalated to 100 mg/m² IV D1, D8 Q3W.

The safety (including DLT), tolerability, and PK are the study endpoints of phase I. Phase II is a multi-center, randomized, open-label, four parallel arms designed study to assess the anti-cancer efficacy, safety and biomarker correlation to efficacy. The therapies in the four treatment arms are: the anti-PD-L1 Ab+afuresertib+nab-paclitaxel; the anti-PD-L1 Ab+nab-paclitaxel; afuresertib+nab-paclitaxel; and nab-paclitaxel monotherapy, respectively. The trial enrollment is a maximum of 21 patients in the Phase I dose escalation part, not counting those that might be replaced. In the Phase II part, 80 patients are randomized with a ratio of 1:1:1:1 to the four open-labelled treatment arms to evaluate efficacy and safety of the four treatments in patients with mTNBC.

In the Phase I dose escalation part, patients who did not have minimum exposure of the triple combination therapy, i.e. missing >25% of the planned doses of afuresertib (<16 days in first cycle) or missing more than one dose of nab-paclitaxel or missing any dose of the anti-PD-L1 Ab and discontinue the trial other than a DLT during the first treatment cycle (21 days) of the Phase I period are replaced. The PK of the anti-PD-L1 antibody and afuresertib are assessed based on plasma levels of the anti-PD-L1 Ab and afuresertib obtained at different time points.

Once the RP2D of the anti-PD-L1 Ab and afuresertib and nab-paclitaxel is selected, a cohort consisting of 80 mTNBC patients who meet the inclusion/exclusion criteria is randomized to each of the four arms in the Phase II to assess the efficacy and safety of the four treatments. The preliminary anti-tumor efficacy of the three combination therapies are assessed by the measurement of ORR as the primary endpoint, whereas PFS, DOR, DCR are the secondary endpoints in phase II study. Patients with biomarkers, such as PTEN/PI3K/AKT/BRCA alternations and PD-L1 level, are tested retrospectively and correlated to the anti-tumor efficacy retrospectively.

### Drug Preparation and Administration

TABLE 13
Treatment and dosing information

| | | | | Route and | |
|---|---|---|---|---|---|
| Intervention | Dosage | Unit Dose | | Duration of | |
| Name | Formulation | Strength(s) | Dose Level(s) | Administration | Sourcing |
| Afuresertib | Tablet | 50 mg, 75 mg | 100 mg and | Oral | Provided centrally |
| | | | 125 mg QD | | by the Sponsor |
| anti-PD-L1 Ab | Solution for | 100 mg/vial | 800 mg and | IV infusion | Provided centrally |
| | injection | | 1200 mg | (30 minutes) | by the Sponsor |
| Nab-paclitaxel | Lyophilized | 100 mg/vial | 125 mg/m² IV | IV infusion | Provided centrally |
| | powder | | D1, D8 Q3W | (30 minutes) | by the Sponsor |

Afuresertib is dosed with approximately 200 mL of water and given prior to the anti-PD-L1 Ab and nab-paclitaxel IV infusion. Patients fast overnight (i.e., at least 8 hours) before dosing. Patients also fast for 2 hours after dosing.

The anti-PD-L1 Ab is administered using IV infusion on Day 1 of each 3-week treatment cycle after all procedures and assessments have been completed. The anti-PD-L1 Ab is administered as a dose of 800 mg or 1200 mg using a 30-minute IV infusion. However, given the variability of infusion pumps from site to site, a window between −5 minutes and +10 minutes is permitted (i.e., infusion time is 30 minutes [−5 min/+10 min]).

Nab-paclitaxel is administered as a 30-minute IV infusion at 125 mg/m² on Day 1 (approximately 30-60 minutes post administration of the anti-PD-L1 Ab) and Day 8 of each 3-week cycle. The infusion rate may be decreased if needed to decrease the severity/frequency of hypersensitivity reactions. Frequent vital sign monitoring is required, as appropriate. Administration of concomitant medications to prevent/treat hypersensitivity reactions follow local medical practice and recommendations included in the product label.

### Efficacy Assessments

### 1. Tumor Imaging and Assessment of Disease

Tumor imaging is acquired by computerized tomography (CT). For the abdomen and pelvis, contrast-enhanced MRI may be used when CT with iodinated contrast is contraindicated, or when mandated by local practice. MRI is the modality for imaging the brain. The same imaging technique regarding modality, ideally the same scanner, and the use of contrast is used in a participant throughout the study to optimize the reproducibility of the assessment of existing and new tumor burden and improve the accuracy of the assessment of response or progression based on imaging. Note: for the purposes of assessing tumor imaging, the term “investigator” refers to the local investigator at the site and/or the radiological reviewer located at the site or at an offsite facility.

### 2. RECIST 1.1 Assessment of Disease

RECIST 1.1 is used as the primary measure for assessment of tumor response, date of disease progression, and as a basis for all protocol guidelines related to disease status (e.g., discontinuation of study treatment). Although RECIST 1.1 references a maximum of 5 target lesions in total and 2 per organ, this protocol allows a maximum of 10 target lesions in total and 5 per organ to enable a broader sampling of tumor burden.

3. iRECIST Assessment of Disease

iRECIST is based on RECIST 1.1 but adapted to account for the unique tumor response seen with immunotherapeutic drugs. iRECIST is used by the Investigator to assess tumor response and progression and make treatment decisions. When clinically stable, participants should not be discontinued until progression is confirmed by the Investigator, working with local radiology, according to the rules outlined in Appendix 1. This allowance to continue treatment despite initial radiologic PD considers the observation that some participants can have a transient tumor flare in the first few months after the start of immunotherapy, and then experience subsequent disease response. This data is captured in the clinical database.

Clinical stability is defined as the following:
Absence of symptoms and signs indicating clinically significant progression of disease
No decline in ECOG performance status
No requirements for intensified management, including increased analgesia, radiation, or other palliative care

Any participant deemed clinically unstable is discontinued from study treatment at site-assessed first radiologic evidence of PD and is not required to have repeat tumor imaging for confirmation of PD by iRECIST.

If the Investigator decides to continue treatment, the participant may continue to receive study treatment and the tumor assessment is repeated 4 to 8 weeks later to confirm PD by iRECIST, per Investigator assessment.

If repeat imaging does not confirm PD per iRECIST, as assessed by the Investigator, and the participant continues to be clinically stable, study treatment may continue and follow the regular imaging schedule. If PD is confirmed, participants is discontinued from study treatment.

If a participant has confirmed radiographic progression (iCPD) as defined in Appendix 1, study treatment is discontinued; however, if the participant is achieving a clinically meaningful benefit, an exception to continue study treatment may be considered following consultation with the Sponsor. In this case, if study treatment is continued, tumor imaging should continue to be performed following the intervals as outlined in Section 7.1.

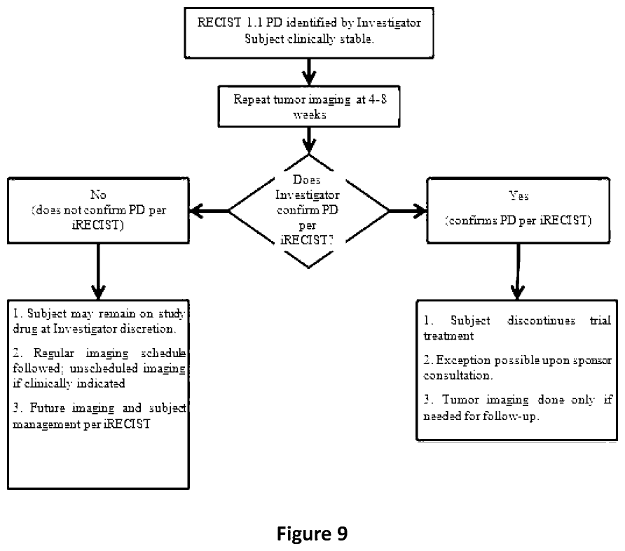

A description of the adaptations and iRECIST process is provided in Appendix 1, with additional details in the iRECIST publication (Seymour L, et al. Lancet Oncol. 2017; 18(3):e143-e152). A summary of imaging and treatment requirements after first radiologic evidence of progression is provided in Table 14 and illustrated as a flowchart in FIG. 9.

TABLE 14
Imaging and Treatment after First Radiologic Evidence of Progressive Disease

| | Clinically Stable | | Clinically Unstable | |
|---|---|---|---|---|
| | Imaging | Treatment | Imaging | Treatment |
| First radiologic | Repeat imaging at | May continue | First radiologic | Discontinue |
| evidence of PD by | 4 to 8 weeks to | study treatment at | evidence of PD by | treatment |
| RECIST 1.1 | confirm PD. | the Investigator's | RECIST 1.1 | |
| | | discretion while | | |
| | | awaiting confirmatory | | |
| | | tumor imaging by | | |
| | | site by iRECIST. | | |
| Repeat tumor imaging | No additional | Discontinue treatment | No additional | Not applicable |
| confirms PD (iCPD) | imaging required. | (exception is possible | imaging required. | |
| by iRECIST per | | upon consultation | | |
| Investigator assessment | | with Sponsor). | | |
| Repeat tumor imaging | Repeat imaging at | Continue study | Repeat imaging at | Discontinue |
| shows iUPD by | 4 to 8 weeks to | treatment at the | 4 to 8 weeks to | treatment. |
| iRECIST per | confirm PD. May | Investigator's | confirm PD per | |
| Investigator assessment | occur at next | discretion. | Investigator's | |
| | regularly scheduled | | discretion | |
| | imaging visit. | | | |
| Repeat tumor imaging | Continue regularly | Continue study | Continue regularly | May restart study |
| shows iSD, iPR, or | scheduled imaging | treatment at the | scheduled imaging | treatment |
| iCR by iRECIST per | assessments. | Investigator's | assessments. | if condition has |
| Investigator assessment. | | discretion. | | improved and/or |
| | | | | clinically stable |
| | | | | per Investigator's |
| | | | | discretion. Next |
| | | | | tumor imaging |
| | | | | should occur |
| | | | | according to the |
| | | | | regular imaging |
| | | | | schedule. |

iCPD = iRECIST confirmed progressive disease;
iCR = iRECIST complete response;
iRECIST = modified Response Evaluation Criteria in Solid Tumors 1.1 for immune-based therapeutics;
iSD = iRECIST stable disease;
iUPD = iRECIST unconfirmed progressive disease;
PD = progressive disease;
RECIST 1.1 = Response Evaluation Criteria in Solid Tumors 1.1.;
VOP = verification of progression
Note:
If disease progression has been verified per iRECIST, further management is performed by the site. If disease progression has not been verified per iRECIST and the condition is clinically stable, ideally the site should continue treatment until progression is verified per iRECIST.

### Definitions

Response is evaluated in this study using the international criteria (version 1.1) proposed by the RECIST Committee (Eisenhauer E A, et al., Eur J Cancer. 2009 January; 45(2):228-47). Changes in only the largest diameter (unidimensional measurement) of the tumor lesions are used in the RECIST 1.1 criteria. Note: Lesions are either measurable or non-measurable using the criteria provided below. The term “evaluable” in reference to measurability is not used because it does not provide additional meaning or accuracy.

### Measurable Disease

Measurable disease is defined by the presence of at least one measurable lesion. Measurable lesions are defined as those that can be accurately measured in at least one dimension [longest diameter (LD) in the plane of measurement to be recorded] with a minimum size of:
10 mm by CT scan (CT scan slice thickness no greater than 5 mm),
10 mm caliper measurement by clinical exam (lesions which cannot be accurately measured with calipers is recorded as non-measurable),
20 mm by chest x-ray

Malignant lymph nodes: To be considered pathologically enlarged and measurable, a lymph node must be ≥15 mm in short axis when assessed by CT scan (CT scan slice thickness no greater than 5 mm).

### Non-Measurable Disease

All other lesions (or sites of disease), including small lesions (LD<10 mm or pathological lymph nodes with ≥10 to <15 mm short axis) are considered non-measurable disease. Lesions considered truly non-measurable include leptomeningeal disease, ascites, pleural/pericardial effusions, lymphangitis cutis/pulmonis, inflammatory breast disease, abdominal masses/abdominal organomegaly identified by physical exam and not followed by CT or MRI.

Bone lesions, cystic lesions and lesions previously treated with local therapy are considered as follows:

Bone lesions:
Bone scan, PET scan or plain films are not considered adequate imaging techniques to measure bone lesions. However, these techniques can be used to confirm the presence or disappearance of bone lesions.
Lytic bone lesions or mixed lytic-blastic lesions, with identifiable soft tissue components, that can be evaluated by cross sectional imaging techniques (i.e., CT or MRI) can be considered as measurable lesions if the soft tissue component meets the definition of measurability described above.
Blastic bone lesions are non-measurable.

### Cystic Lesions:

Lesions that meet the criteria for radiographically defined simple cysts should not be considered malignant lesions (neither measurable or non-measurable) since they are, by definition, simple cysts.
‘Cystic lesions’ thought to represent cystic metastases can be considered measurable lesions, if they meet the definition of measurability described above. However, if non cystic lesions are present in the same patient, these are preferred for selection as target lesions.
   Lesions with Prior Local Treatment:
Tumor lesions situated in a previously irradiated area, or in an area subjected to other loco-regional therapy, are usually not considered measurable unless there has been demonstrated progression in the lesion.

### Target Lesions

All measurable lesions up to a maximum of two lesions per organ and five lesions in total, representative of all involved organs, is identified as target lesions and recorded and measured at baseline. Target lesions are selected based on their size (lesions with the longest diameter) and their suitability for accurate repeated measurements (either by imaging techniques or clinically). A sum of the diameters (longest for non-nodal lesions, short axis for nodal lesions) for all target lesions is calculated and reported as the baseline sum diameters. The baseline sum diameters is used as reference by which to characterize the objective tumor response.

### Lymph Node Assessment

For lymph nodes, measurements are made of the short axis, which is defined as perpendicular to the LD of node assessed in the plane of measurement:
Target lesion if short axis ≥15 mm
Non-target lesion if short axis is ≥10 but <15 mm
Normal if short axis <10 mm

For baseline, add the actual short axis measurement to the sum of LD of non-nodal lesions.

### Non-Target Lesions

All other lesions (or sites of disease) including pathological lymph nodes are identified as non-target lesions and should also be recorded at baseline. Measurements of these lesions are not required, and these lesions are followed as “present,” “absent,” or in rare cases “unequivocal progression.” In addition, it is possible to record multiple non-target lesions involving the same organ as a single item on the eCRF (e.g., ‘multiple enlarged pelvic lymph nodes’ or ‘multiple liver metastases’).

### Guidelines for Evaluation of Measurable Disease

All measurements are taken and recorded in metric notation using a ruler or calipers. All baseline evaluations are performed as closely as possible to the beginning of treatment and never more than 4 weeks before the beginning of the treatment.

The same method of assessment and the same technique are used to characterize each identified and reported lesion at baseline and during follow-up. Imaging-based evaluation is preferred to evaluation by clinical examination when both methods have been used to assess the antitumor effect of a treatment.

Clinical lesions. Clinical lesions is only considered measurable when they are superficial and ≥10 mm diameter as assessed using calipers (e.g., skin nodules). In the case of skin lesions, documentation by color photography, including a ruler to estimate the size of the lesion, is recommended. When lesions can be evaluated by both clinical exam and imaging, imaging evaluation is undertaken since it is more objective and may be reviewed at the end of the study.

Chest x-ray. Chest CT is preferred over chest x-ray, particularly when progression is an important endpoint. Lesions on chest x-ray may be considered measurable if they are clearly defined and surrounded by aerated lung.

Conventional CT and MRI. This guideline has defined measurability of lesions on CT scan based on the assumption that CT slice thickness is 5 mm or less. When CT scans have slice thickness >5 mm, the minimum size for a measurable lesion is twice the slice thickness. MRI is acceptable in certain situations (e.g., for body scans).

Ultrasound. Ultrasound should not be used to measure tumor lesions. Ultrasound examinations cannot be reproduced in their entirety for independent review at a later date because they are operator dependent. If new lesions are identified by ultrasound, confirmation by CT or MRI is advised. If there is concern about radiation exposure at CT, MRI may be used instead of CT.

Endoscopy, Laparoscopy. The utilization of these techniques for objective tumor evaluation is not advised. However, such techniques can be useful to confirm complete pathological response when biopsies are obtained or to determine relapse in trials where recurrence following complete response or surgical resection is an endpoint.

Tumor markers. Tumor markers alone cannot be used to assess objective tumor response. If markers are initially above the upper normal limit, they must normalize for a patient to be considered in complete clinical response.

Cytology, Histology. These techniques can be used to differentiate between PR and CR in rare cases (e.g., residual lesions in tumor types such as germ cell tumors, where known residual benign tumors can remain).

### Response Evaluation Criteria (RECIST v1.1)

TABLE 15
Evaluation of Target Lesions

| | |
|---|---|
| Complete | Disappearance of all target lesions. Any pathological |
| Response (CR): | lymph nodes (whether target or non-target) must |
| | have reduction in short axis to <10 mm. |
| Partial | At least a 30% decrease in the sum of diameters of |
| Response (PR): | target lesions, taking as reference the baseline |
| | sum diameters. |
| Progressive | At least a 20% increase in the sum of diameters of |
| Disease (PD): | target lesions, taking as reference the smallest sum |
| | on study (this includes the baseline sum if that is the |
| | smallest). In addition to the relative increase of 20%, |
| | the sum must also demonstrate an absolute increase |
| | of at least 5 mm. The appearance of one or more |
| | new lesions is also considered progression. |
| Stable | Neither sufficient shrinkage to qualify for PR nor |
| Disease (SD): | sufficient increase to qualify for PD, taking as |
| | reference the smallest sum diameters while on study. |

### Assessment of Target Lymph Nodes

Lymph nodes identified as target lesions should always have the actual short axis measurement recorded (measured in the same anatomical plane as the baseline exam), even if the nodes regress to below 10 mm on study. In order to qualify for CR, each node must achieve a short axis <10 mm. For PR, SD and PD, the actual short axis measurement of the nodes is to be included in the sum of target lesions.

Target Lesions that Become “Too Small to Measure”

All lesions (nodal and non-nodal) recorded at baseline should have their actual measurements recorded at each subsequent evaluation, even when very small (e.g., 2 mm). If it is the opinion of the radiologist that the lesion has disappeared, the measurement is recorded as 0 mm. If the lesion is believed to be present and is faintly seen but too small to measure, a default value of 5 mm is assigned.

Lesions that Split or Coalesce on Treatment

When non-nodal lesions fragment, the longest diameters of the fragmented portions is added together to calculate the target lesion sum. Similarly, as lesions coalesce, a plane between them may be maintained that would aid in obtaining diameter measurements of each individual lesion. If the lesions have truly coalesced, the vector of the longest diameter is the maximal longest diameter for the ‘coalesced lesion.’

TABLE 16
Evaluation of Non-target Lesions

| | |
|---|---|
| Complete | Disappearance of all non-target lesions and |
| Response (CR): | normalization of tumor marker level. All |
| | lymph nodes must be non-pathological in size |
| | (<10 mm short axis). |
| Non-CR/Non-PD: | Persistence of one or more non-target lesion(s) |
| | and/or maintenance of tumor marker level |
| | above the normal limits. |
| Progressive | Unequivocal progression of existing non-target |
| Disease (PD): | lesions. (The appearance of one or more new |
| | lesions is also considered progression.) To |
| | achieve ‘unequivocal progression’ on the basis |
| | of the non-target disease, there must be an |
| | overall level of substantial worsening in |
| | non-target disease such that, even in the |
| | presence of SD or PR in target disease, the |
| | overall tumor burden has increased sufficiently |
| | to merit discontinuation. |

### New Lesions

The finding of a new lesion is unequivocal (i.e., not attributed to differences in scanning technique, change in imaging modality, or findings thought to represent something other than tumor, such as a ‘new’ healing bone lesion). A lesion identified on a follow-up study in an anatomical location that was not scanned at baseline is considered a new lesion and indicates disease progression. If a new lesion is equivocal, continued therapy and follow-up evaluation clarifies if it represents truly new disease. If repeat scans confirm this is definitely a new lesion, then progression is declared using the date of the initial scan.

### Evaluation of Best Overall Response

The best overall response (BOR) is the best response recorded from the start of the treatment until disease progression/recurrence (taking as reference for progressive disease the smallest measurements recorded since the treatment started). The patient's BOR assignment depends on findings of both target and non-target disease and also takes into consideration the appearance of new lesions. Furthermore, depending on the nature of the study, it may also require confirmatory measurement. Specifically, in non-randomized trials where response is the primary endpoint, confirmation of PR or CR is needed to deem either one the “BOR.”

It is assumed that at each protocol-specified time point, a response assessment occurs. Table 17 provides a summary of the overall response status calculation at each time point for patients who have measurable disease at baseline. When patients have non-measurable disease, Table 18 is used.

TABLE 17
Time point response: Patients with target (+/− non-target) disease

| Target | Non-target | New | Overall |
|---|---|---|---|
| Lesions | Lesions | Lesions | Response |
| CR | CR | No | CR |
| CR | Non-CR/non-PD | No | PR |
| CR | Not evaluated | No | PR |
| PR | Non-PD or not | No | PR |
| | all evaluated | | |
| SD | Non-PD or not | No | SD |
| | all evaluated | | |
| Not all | Non-PD | No | NE |
| evaluated | | | |
| PD | Any | Yes or No | PD |
| Any | PD | Yes or No | PD |
| Any | Any | Yes | PD |

CR = complete response,
PR = partial response,
SD = stable disease
PD = progressive disease,
NE = inevaluable

TABLE 18
Time point response: Patients with non-target disease only

| | New | |
|---|---|---|
| Non-target Lesions | Lesions | Overall Response |
| CR | No | CR |
| Non-CR/non-PD | No | Non-CR/non-PD* |
| Not all evaluated | No | NE |
| Unequivocal PD | Yes or No | PD |
| Any | Yes | PD |

CR = complete response;
PD = progressive disease;
NE = inevaluable
*Non-CR/non-PD is preferred over SD for non-target disease

Best response determination for studies where confirmation of CR or PR is required: Complete or partial responses may be claimed only if the criteria for each are confirmed by a repeat assessment approximately 4 weeks later (generally 4 weeks). In this circumstance, the BOR can be interpreted as in Table 19.

TABLE 19
Best overall response when confirmation of CR and PR required

| Overall | Overall | |
|---|---|---|
| Response | Response | |
| First time | Subsequent | |
| point | time point | Best overall response |
| CR | CR | CR |
| CR | PR | SD, PD or PR* |
| CR | SD | SD provided minimum criteria for |
| | | SD duration met, otherwise PD |
| CR | PD | SD provided minimum criteria for |
| | | SD duration met, otherwise PD |
| CR | NE | SD provided minimum criteria for |
| | | SD duration met, otherwise NE |
| PR | CR | PR |
| PR | PR | PR |
| PR | SD | SD |
| PR | PD | SD provided minimum criteria for |
| | | SD duration met, otherwise PD |
| PR | NE | SD provided minimum criteria for |
| | | SD duration met, otherwise NE |
| NE | NE | NE |

CR = complete response;
PR = partial response;
SD = stable disease;
PD = progressive disease;
NE = inevaluable
*If CR is truly met at first time point, then any disease seen at a subsequent time point, even disease meeting PR criteria relative to baseline, makes the disease PD at that point (since disease must have reappeared after CR). Best response would depend on whether minimum duration for SD was met. However, sometimes ‘CR’ may be claimed when subsequent scans suggest small lesions were likely still present and, in the fact patient had PR, not CR, at the first time point. Under these circumstances, the original CR is changed to PR and the best response is PR.

### Confirmatory Measurement/Duration of Response

### Confirmation

To be assigned a status of PR, CR, or PD, changes in tumor measurements must be confirmed by repeat assessments that are performed 4 weeks after the criteria for response are first met. In the case of SD, follow-up measurements must have met the SD criteria at least once after study entry at a minimum interval of 6-8 weeks. Confirmation of PD must follow iRECIST process for Assessment of Disease Progression (Appendix 1).

### Duration of Overall Response

The duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that recurrent disease is objectively documented.

### Duration of Stable Disease

SD is measured from the start of the treatment until the criteria for progression are met, taking as reference the smallest measurements recorded since the treatment started.

### Progression-Free Survival

PFS is defined as the duration of time from start of treatment to time of progression.

### Analysis of the Primary Endpoint:

The primary endpoint of efficacy is ORR. The primary analysis of ORR is based on tumor assessment results reviewed by investigators according to RECIST 1.1. For each treatment arm, the Clopper-Pearson method is used to provide an estimation of ORR with a 90% confidence interval (CI). These analyses on ORR are performed for the FAS as primary analysis and for PPS as secondary analysis. The same analysis is performed using the tumor assessment results according to iRECIST for the exploratory purpose if they are different from the those based on RECIST 1.1.

### Analysis of the Secondary Endpoints:

For each treatment arm, BOR according to RECIST 1.1 is summarized. The Clopper-Pearson method is used to provide an estimation of the rate of each BOR category (CR, PR, SD and PD) and DCR with 90% CIs. Change from baseline in tumor burden is presented graphically, using waterfall plot.

For each individual arm, when the number of evaluable patients is not less than 10, DOR (when the number of responders is not less than 10) and PFS is analyzed using the Kaplan-Meier method. The minimum, maximum and medians (along with the 90% CIs) is provided.

### Example 5: Preliminary Safety and Efficacy of the Combination Therapy with Anti-PD-L1 Antibody+Afuresertib+Nab-Paclitaxel in Advanced Solid Tumors, Primarily in Triple-Negative Breast Cancer (TNBC)

The phase I/II study (NCT05390710) was initiated on Jun. 12, 2021. This is a multi-center, open-label, dose-escalation and proof-of-concept study. In phase I of the study, patients with advanced solid tumors (TNBC preferred) who have failed 0 to 3 lines of standard treatment are eligible. Efficacy evaluation is based on RECIST 1.1. Dose escalation used Bayesian optimal interval (BOIN) design and was reviewed by Safety Review Committee (SRC).

21 patients were enrolled in the study. Among the 21 patients, the median prior lines of standard treatment was 1 (ranging from 0 to 3). Six patients (3 TNBC, 2 hormone-receptor-positive (HR+) breast cancer, 1 lung adenocarcinoma) were enrolled in cohort 1, which received the following dosing: afuresertib 100 mg PO QD+LAE005 1200 mg IV Q3W+nab-paclitaxel 125 mg/m2 IV D1, D8, Q3W. Five TNBC patients were enrolled in cohort 2, which received the following dosing: afuresertib 100 mg PO QD+LAE005 1200 mg IV Q3W+nab-paclitaxel 100 mg/m2 IV D1, D8, Q3W. Ten TNBC patients were enrolled in cohort 3, which received the following dosing: afuresertib 125 mg PO QD+LAE005 1200 mg IV Q3W+nab-paclitaxel 100 mg/m2 IV D1, D8, Q3W. Two dose-limiting toxicity (DLT) cases were reported out of the 6 and 7 DLT-evaluable patients in cohorts 1 and 3, respectively. The DLT cases exhibited grade 3 rash (rash maculopapular and erythema multiforme). No DLT cases were observed out of 5 DLT evaluable patients in cohort 2. The most common treatment-emergent adverse events (AEs) were white blood cell count decreased (85.7%), neutrophil count decreased (81.0%) and rash (71.4%). The most common grade 3 or above AEs were white blood cell count decreased (38.1%), neutrophil count decreased (38.1%), rash (19.0%) and lymphocyte count decreased (19.0%). Most of the AEs are manageable, reversible, and recovered after routine treatments. Five partial responses (PR) (4 confirmed) were observed among 12 TNBC patients with at least 1 tumor assessment. The overall response rate (ORR) is ½, ⅔ and 1/7 in cohort 1, 2, 3, respectively.

In conclusion, the triplet regimen shows a manageable safety profile and encouraging preliminary activity in TNBC patients. This triplet regimen warrants further investigation.

1. A method of treating cancer in a subject, comprising administering to the subject in need thereof an effective amount of:
(i) N-{(1S)-2-amino-1-[(3-fluorophenyl)methyl]ethyl}-5-chloro-4-(4-chloro-1-methyl-1H-pyrazol-5-yl)-2-thiophenecarboxamide (afuresertib), or a pharmaceutically acceptable salt thereof; and
(ii) and an isolated antibody molecule capable of binding to human Programmed Death-Ligand 1 (PD-L1) (anti-PD-L1 antibody).

2. The method of claim 1, wherein the afuresertib, or a pharmaceutically acceptable salt, thereof is administered to the subject in a total daily dosage of from about 25 mg to about 200 mg, on a free base basis; a total daily dosage of from about 50 mg to about 200 mg, on a free base basis; a total daily dosage of from about 75 mg to about 150 mg, on a free base basis; a total daily dosage of about 100 mg to about 125 mg, on a free base basis; a total daily dosage of about 100 mg, on a free base basis; a total daily dosage of about 110 mg, on a free base basis; a total daily dosage of about 115 mg, on a free base basis; a total daily dosage of about 120 mg, on a free base basis; or in a total daily dosage of about 125 mg, on a free base basis.

3.-5. (canceled)

6. The method of claim 1, wherein the afuresertib, or a pharmaceutically acceptable salt thereof, is administered to the subject in a total daily dosage of about 100 mg, on a free base basis.

7.-9. (canceled)

10. The method of claim 1, wherein the afuresertib, or a pharmaceutically acceptable salt thereof, is administered to the subject in a total daily dosage of about 125 mg, on a free base basis.

11. The method of claim 1, wherein the afuresertib, or the pharmaceutically acceptable salt thereof, is administered to the subject once daily (QD).

12. The method of claim 1, wherein the afuresertib, or the pharmaceutically acceptable salt thereof, is administered orally.

13. The method of claim 1, wherein the afuresertib, or the pharmaceutically acceptable salt thereof, is formulated as part of a pharmaceutically acceptable composition further comprising one or more pharmaceutically acceptable excipients.

14. The method of claim 1, wherein the anti-PD-L1 antibody is selected from Atezolizumab, Durvalumab and an antibody comprising:
(i) (a) a heavy chain variable region (VH) comprising a VHCDR1 amino acid sequence chosen from SEQ ID NO: 1, SEQ ID NO: 7 or SEQ ID NO: 9; a VHCDR2 amino acid sequence of SEQ ID NO: 2; and a VHCDR3 amino acid sequence of SEQ ID NO: 3; and (b) a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
or
(ii) (a) a heavy chain variable region (VH) comprising a VHCDR1 amino acid sequence chosen from SEQ ID NO: 1, SEQ ID NO: 7 or SEQ ID NO: 9; a VHCDR2 amino acid sequence of SEQ ID NO: 8; and a VHCDR3 amino acid sequence of SEQ ID NO: 3; and (b) a light chain variable region (VL) comprising a VLCDR1 amino acid sequence of SEQ ID NO: 10, a VLCDR2 amino acid sequence of SEQ ID NO: 11, and a VLCDR3 amino acid sequence of SEQ ID NO: 12.

15. The method of claim 1, wherein the anti-PD-L1 antibody comprises a variable heavy (VH) chain comprising the amino acid sequence set forth in any one of SEQ ID NOs: 13-16.

16. The method of claim 1, wherein the anti-PD-L1 antibody comprises a variable light (VL) chain comprising the amino acid sequence set forth in any one of SEQ ID NOs: 17-20.

17. The method of claim 1, wherein the anti-PD-L1 antibody comprises a heavy chain comprising the amino acid sequence set forth in any one of SEQ ID NOs: 21-24.

18. The method of claim 1, wherein the anti-PD-L1 antibody comprises a light chain comprising the amino acid sequence set forth in any one of SEQ ID NOs: 25-28.

19. The method of claim 1, wherein the anti-PD-L1 antibody is administered to the subject once every three weeks at a dosage of about 500 mg to about 2000 mg, a dosage of about 500 mg to about 1500 mg, a dosage of about 800 mg to about 1200 mg, a dosage of about 800 mg, a dosage of about 900 mg, a dosage of about 1000 mg, a dosage of about 1100 mg, or a dosage of about 1200 mg.

20.-25. (canceled)

26. The method of claim 1, wherein the anti-PD-L1 antibody is administered to the subject once every three weeks at a dosage of about 1200 mg.

27. The method of claim 1, wherein the treating further comprises administering a chemotherapeutic agent.

28. The method of claim 27, wherein the chemotherapeutic agent is selected from a platinum-based agent, a taxane, an epothilone, an anti-microtubule agent, an immunomodulatory agent, and a proteasome inhibitor.

29. The method of claim 27, wherein the chemotherapeutic agent is paclitaxel or nab-paclitaxel.

30. The method of claim 29, wherein the paclitaxel or nab-paclitaxel is administered to the subject on day 1 and day 8+/−1 day of a 3 weeks+/−3 days treatment cycle at a dosage of about 75 to about 200 mg/m².

31. The method of claim 29, wherein the paclitaxel or nab-paclitaxel is administered to the subject on day 1 and day 8+/−1 day of a 3 weeks+/−3 days treatment cycle at a dosage of about 125 mg/m².

32. The method of claim 1, wherein the method comprises the following administration regimen per treatment cycle:
(a) on day 1 of the treatment cycle:
(i) administering afuresertib, or a pharmaceutically acceptable salt thereof; and
(ii) after completion of step (a)(i), administering the anti-PD-L1 antibody; and
(b) administering the afuresertib, or a pharmaceutically acceptable salt thereof, daily starting on day 2 of the treatment cycle;
wherein the duration of each treatment cycle is 3 weeks+/−3 days long.

33. The method of claim 1, wherein the method comprises the following administration regimen per treatment cycle:
(a) on day 1 of the treatment cycle:
(i) administering about 25 to about 200 mg/kg of the afuresertib, or a pharmaceutically acceptable salt thereof, on a free base basis; and
(ii) after completion of step (a)(i), administering about 500 to about 1500 mg of the anti-PD-L1 antibody; and
(b) administering about 100 to about 125 mg/kg of the afuresertib, or a pharmaceutically acceptable salt thereof, on a free base basis daily starting on day 2 of the treatment cycle;
wherein the duration of each treatment cycle is 3 weeks+/−3 days.

34. The method of claim 32, wherein the administration regimen repeats for at least 8 cycles.

35. The method of claim 32, further comprising administering paclitaxel or nab-paclitaxel on days 1 and 8 of the treatment cycle.

36. The method of claim 32, wherein paclitaxel or nab-paclitaxel is administered after the administration of the anti-PD-L1 antibody on day 1 of the treatment cycle and after the administration of afuresertib, or a pharmaceutically acceptable salt thereof, on day 8 of the treatment cycle.

37. The method of claim 35, wherein about 75 to about 125 mg/m² of paclitaxel or nab-paclitaxel is administered on day 1 of the treatment cycle.

38. The method of claim 35, wherein about 30 minutes to about 60 minutes after administration of afuresertib, or a pharmaceutically acceptable salt thereof, about 75 to about 125 mg/m² of paclitaxel or nab-paclitaxel was administered on day 8 of the treatment cycle.

39. The method of claim 1, wherein the anti-PD-L1 antibody is administered to the subject orally, parenterally, subcutaneously, intravenously, rectally, intramuscularly, intraperitoneally, intranasally, transdermally, or by inhalation or intracavitary installation, topically, or by application to mucous membranes.

40.-42. (canceled)

43. The method of claim 1, wherein:
the afuresertib, or the pharmaceutically acceptable salt thereof, and the anti-PD-L1 antibody are administered sequentially; or
the afuresertib, or the pharmaceutically acceptable salt thereof, and the anti-PD-L1 antibody are administered simultaneously.

44. (canceled)

45. The method of claim 1, wherein the method comprises the following administration regimen per treatment cycle:
(a) on day 1 of the treatment cycle:
(i) orally administering about 100 to about 125 mg/kg of the afuresertib, or a pharmaceutically acceptable salt thereof, on a free base basis;
(ii) after completion of step (a)(i), intravenously administering about 800 to about 1200 mg of the anti-PD-L1 antibody; and
(iii) about 30 minutes to about 60 minutes after completion of step (a)(ii), intravenously administering about 125 mg/m² mg/kg of nab-paclitaxel;
(b) orally administering about 100 to about 125 mg/kg of the afuresertib, or a pharmaceutically acceptable salt thereof, on a free base basis, on days 2-7 and 9-21 (+/−3 days) of the treatment cycle; and
(c) on day 8 of the treatment cycle:
(i) orally administering about 100 to about 125 mg/kg of the afuresertib, or a pharmaceutically acceptable salt thereof, on a free base basis; and
(ii) after completion of step (c)(i), intravenously administering about 125 mg/m² of nab-paclitaxel;
wherein each treatment cycle is 3 weeks+/−3 days long and the administration regimen repeats for at least 8 cycles.

46. The method of claim 1, wherein the treating increases at least one of the following parameters in the subject relative to a control subject population treated with paclitaxel or nab-paclitaxel monotherapy alone:
(a) objective response rate (ORR);
(b) best overall response rate (BOR);
(c) progression free survival (PFS);
(d) disease control rate (DCR);
(e) duration of response (DoR); and
(f) overall survival (OS).

47. The method of claim 1, wherein the cancer is a solid tumor.

48. The method of claim 1, wherein the cancer is selected from a lung cancer, a squamous cell lung cancer, a melanoma, a renal cancer, a liver cancer, a myeloma, a prostate cancer, a breast cancer, an ER+ breast cancer, an IM-TN breast cancer, a colorectal cancer, a colorectal cancer with high microsatellite instability, an EBV+ gastric cancer, a pancreatic cancer, a thyroid cancer, a nasopharyngeal cancer, (e.g., differentiated or undifferentiated metastatic or locally recurrent nasopharyngeal carcinoma), a hematological cancer, a non-Hodgkin lymphoma, a leukemia, and a metastatic lesion of the cancer.

49. The method of claim 1, wherein the cancer is a triple negative breast cancer (TNBC) or gastric cancer.

50. A kit for the treatment of cancer, the kit comprising, in separate containers,
(a) a first pharmaceutical composition comprising afuresertib, or a pharmaceutically acceptable salt thereof;
and
(b) a second pharmaceutical composition comprising an anti-PD-L1 antibody.

51.-52. (canceled)

53. The method of claim 1, wherein the afuresertib, or a pharmaceutically acceptable salt thereof, is afuresertib hydrochloride.

54.-59. (canceled)


SEQUENCE LISTING (28 sequences)

SEQ ID NO: 1 - protein, 10 aa, synthetic construct
  REGION 1..10: note = source = /note="Description of Artificial Sequence: Synthetic peptide"
GYTFTSYWMY

SEQ ID NO: 2 - protein, 17 aa, synthetic construct
  REGION 1..17: note = source = /note="Description of Artificial Sequence: Synthetic peptide"
RIDPNSGSTKYNEKFKN

SEQ ID NO: 3 - protein, 11 aa, synthetic construct
  REGION 1..11: note = source = /note="Description of Artificial Sequence: Synthetic peptide"
DYRKGLYAMDY

SEQ ID NO: 4 - protein, 11 aa, synthetic construct
  REGION 1..11: note = source = /note="Description of Artificial Sequence: Synthetic peptide"
KASQDVGTAVA

SEQ ID NO: 5 - protein, 7 aa, synthetic construct
  REGION 1..7: note = source = /note="Description of Artificial Sequence: Synthetic peptide"
WASTRHT

SEQ ID NO: 6 - protein, 9 aa, synthetic construct
  REGION 1..9: note = source = /note="Description of Artificial Sequence: Synthetic peptide"
QQYNSYPLT

SEQ ID NO: 7 - protein, 5 aa, synthetic construct
  REGION 1..5: note = source = /note="Description of Artificial Sequence: Synthetic peptide"
SYWMY

SEQ ID NO: 8 - protein, 6 aa, synthetic construct
  REGION 1..6: note = source = /note="Description of Artificial Sequence: Synthetic peptide"
DPNSGS

SEQ ID NO: 9 - protein, 7 aa, synthetic construct
  REGION 1..7: note = source = /note="Description of Artificial Sequence: Synthetic peptide"
GYTFTSY

SEQ ID NO: 10 - protein, 7 aa, synthetic construct
  REGION 1..7: note = source = /note="Description of Artificial Sequence: Synthetic peptide"
SQDVGTA

SEQ ID NO: 11 - skipped

SEQ ID NO: 12 - protein, 6 aa, synthetic construct
  REGION 1..6: note = source = /note="Description of Artificial Sequence: Synthetic peptide"
YNSYPL

SEQ ID NO: 13 - protein, 120 aa, synthetic construct
  REGION 1..120: note = source = /note="Description of Artificial Sequence: Synthetic polypeptide"
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 14 - protein, 120 aa, synthetic construct
  REGION 1..120: note = source = /note="Description of Artificial Sequence: Synthetic polypeptide"
QVQLVQSGAEVKKPGASVKVSCKASGYTFTSYWMYWVRQATGQGLEWMGRIDPNSGSTKYNEKFKNRFTISRDDSKNTAYLQMNSLKTEDTAVYYCARDYRKGLYAMDYWGQGTTVTVSS

SEQ ID NO: 15 - protein, 120 aa, synthetic construct
  REGION 1..120: note = source = /note="Description of Artificial Sequence: Synthetic polypeptide"
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 16 - protein, 120 aa, synthetic construct
  REGION 1..120: note = source = /note="Description of Artificial Sequence: Synthetic polypeptide"
EVQLVQSGAEVKKPGESLRISCKGSGYTFTSYWMYWVRQAPGQGLEWMGRIDPNSGSTKYNEKFKNRVTISVDTSKNQFSLKLSSVTAADTAVYYCARDYRKGLYAMDYWGQGTTVTVSS

SEQ ID NO: 17 - protein, 107 aa, synthetic construct
  REGION 1..107: note = source = /note="Description of Artificial Sequence: Synthetic polypeptide"
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 18 - protein, 107 aa, synthetic construct
  REGION 1..107: note = source = /note="Description of Artificial Sequence: Synthetic polypeptide"
DIVMTQTPLSLPVTPGEPASISCKASQDVGTAVAWYLQKPGQSPQLLIYWASTRHTGIPARFSGSGSGTEFTLTISSLQSEDFAVYYCQQYNSYPLTFGQGTKVEIK

SEQ ID NO: 19 - protein, 107 aa, synthetic construct
  REGION 1..107: note = source = /note="Description of Artificial Sequence: Synthetic polypeptide"
EIVLTQSPDFQSVTPKEKVTITCKASQDVGTAVAWYLQKPGQSPQLLIYWASTRHTGVPDRFSGSGSGTDFTLKISRVEAEDVGVYYCQQYNSYPLTFGQGTKVEIK

SEQ ID NO: 20 - protein, 107 aa, synthetic construct
  REGION 1..107: note = source = /note="Description of Artificial Sequence: Synthetic polypeptide"
EIVLTQSPDFQSVTPKEKVTITCKASQDVGTAVAWYLQKPGQSPQLLIYWASTRHTGVPSRFSGSGSGTDFTFTISSLQPEDIATYYCQQYNSYPLTFGQGTKVEIK

SEQ ID NO: 21 - protein, 446 aa, synthetic construct
  REGION 1..446: note = source = /note="Description of Artificial Sequence: Synthetic polypeptide"
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 22 - protein, 447 aa, synthetic construct
  REGION 1..447: note = source = /note="Description of Artificial Sequence: Synthetic polypeptide"
QVQLVQSGAEVKKPGASVKVSCKASGYTFTSYWMYWVRQATGQGLEWMGRIDPNSGSTKYNEKFKNRFTISRDDSKNTAYLQMNSLKTEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 447 aa, synthetic construct
  REGION 1..447: note = source = /note="Description of Artificial Sequence: Synthetic polypeptide"
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 446 aa, synthetic construct
  REGION 1..446: note = source = /note="Description of Artificial Sequence: Synthetic polypeptide"
EVQLVQSGAEVKKPGESLRISCKGSGYTFTSYWMYWVRQAPGQGLEWMGRIDPNSGSTKYNEKFKNRVTISVDTSKNQFSLKLSSVTAADTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 25 - protein, 214 aa, synthetic construct
  REGION 1..214: note = source = /note="Description of Artificial Sequence: Synthetic polypeptide"
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 214 aa, synthetic construct
  REGION 1..214: note = source = /note="Description of Artificial Sequence: Synthetic polypeptide"
DIVMTQTPLSLPVTPGEPASISCKASQDVGTAVAWYLQKPGQSPQLLIYWASTRHTGIPARFSGSGSGTEFTLTISSLQSEDFAVYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 214 aa, synthetic construct
  REGION 1..214: note = source = /note="Description of Artificial Sequence: Synthetic polypeptide"
EIVLTQSPDFQSVTPKEKVTITCKASQDVGTAVAWYLQKPGQSPQLLIYWASTRHTGVPDRFSGSGSGTDFTLKISRVEAEDVGVYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 214 aa, synthetic construct
  REGION 1..214: note = source = /note="Description of Artificial Sequence: Synthetic polypeptide"
EIVLTQSPDFQSVTPKEKVTITCKASQDVGTAVAWYLQKPGQSPQLLIYWASTRHTGVPSRFSGSGSGTDFTFTISSLQPEDIATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC